(19) United States
(12) Patent Application Publication
(10) Pub. No.: US 2024/0384260 A1
(43) Pub. Date: Nov. 21, 2024

(54) METHODS OF IN SITU TOTAL RNA-BASED TRANSCRIPTOME PROFILING FOR LARGE-SCALE SUBCELLULAR STRUCTURE PROFILING

(71) Applicant: Baylor College of Medicine, Houston, TX (US)

(72) Inventors: Chenghang Zong, Houston, TX (US); Muchun Niu, Houston, TX (US)

(73) Assignee: Baylor College of Medicine, Houston, TX (US)

(21) Appl. No.: 18/687,261

(22) PCT Filed: Sep. 1, 2022
(86) PCT No.: PCT/US22/75835

§ 371 Date: Feb. 27, 2024

Related U.S. Application Data
(60) Provisional application No. 63/240,339, filed on Sep. 2, 2021.

(51) Int. Cl. C12N 15/10

(52) U.S. Cl. CPC C12N 15/1065

(57) ABSTRACT

Embodiments of the disclosure include high-throughput profiling of transcriptomes of subcellular compartments or structures, including a droplet-based single-cell total-RNA-seq method that enables profiling of transcripts localized in particular subcellular compartment or structures. In specific embodiments, the disclosure provides for transcriptome profiling of single nuclei that allows for construction of a cell atlas using only long non-coding RNA species that can be applied for tissue-wide identification of cell-type-specific lncRNA species.

This application claims priority to U.S. Provisional Patent Application Ser. No. 63/240,339, filed Sep. 2, 2021, which is incorporated by reference herein in its entirety.

### TECHNICAL FIELD

Embodiments of the disclosure include at least the fields of nucleic acid amplification, nucleic acid manipulation, genetics, medicine, and so forth.

### BACKGROUND

Synapses are crucial structures that mediate signal transmission between neurons in complex neural circuits. Advances in microscopy and electrophysiology techniques have unveiled the morphological and electrophysiological heterogeneity existing among individual synapses¹⁻⁵. To facilitate the characterization of synaptic heterogeneity and the construction of a synapse transcriptome atlas, a high-throughput transcriptome profiling method of individual synaptosomes is greatly desired. However, in order to achieve successful profiling of gene expression in individual synaptosomes, new technical features of transcriptome profiling beyond the state-of-art single cell RNA (scRNA)-seq platforms are required. First, individual synaptosomes contain smaller quantities of RNA molecules than single cells or single nuclei. Therefore, a high-sensitivity single-subcellular structure RNA-seq (sssRNA-seq) assay is desired. Second, after synaptosomes are prepared, the materials require immediate fixation to prevent significant leakage of RNA molecules in downstream steps. Hence, RNA-seq chemistry compatible with fixed samples is demanded. Third, to characterize locally spliced genes in the synapses, a total-RNA-based assay that permits simultaneous detection of both mature and nascent RNA is desired.

The present disclosure satisfies a long-felt need in the art in need of transcriptome profiling for large-scale subcellular structure profiling.

### BRIEF SUMMARY

Embodiments of the present disclosure relate in general to methods and compositions for producing DNA libraries representative of RNA sequences of any kind, including at least mRNA, nascent RNA, and long non-coding RNA. In specific embodiments, the disclosure concerns amplifying transcriptomic sequences in situ, such as the transcriptome in fixed subcellular structures or particles.

In particular embodiments, methods are provided herein for the production of amplifiable cDNA in situ from total RNA templates (including rRNA, mRNA, nascent RNA, microRNA, long non-coding RNA, etc.), such as transcript templates, in biological and clinical samples. The in situ-generated cDNA can be barcoded during further amplification to achieve single-subcellular-compartment transcriptome profiling or spatial transcriptome profiling. The methods of the disclosure are adaptable to any small reaction volumes such as nanoliter droplet platform or microwells or other scales of volumes, to generate the total RNA-based transcriptome of up to millions of single subcellular structures, condensates, or particles. The methods of the disclosure are adaptable to platforms carrying primers with regional specific barcodes to generate the total RNA-based transcriptome with spatial resolution.

Embodiments of the disclosure include methods of producing a library representing RNA related to a subcellular structure, comprising the steps of: (a) fixing cellular material (fresh, frozen, or was previously frozen) that is or comprises one or more subcellular structures such that RNA associated with the structure, including nascent RNA, microRNA, long non-coding RNA, and/or mRNA, is affixed to the structure; (b) subjecting the subcellular structures and the RNA to first primers to generate a collection of first complementary polynucleotides that are complementary to one or more different regions in the RNA, thereby producing hybrid molecules between the RNA and first complementary polynucleotides, said hybrid molecules being associated with the subcellular structures, wherein at least one of the first primers comprise random sequence, or random sequence of only three types of nucleotides, or random sequence of only two types of nucleotides and an adaptor; (c) generating a common tail sequence on a 3′ end of the first complementary polynucleotides in the hybrid molecules, wherein said common tail sequence is complementary to a second primer; (d1) encapsulating the subcellular structures and RNA in microscopic volume or microscope volume compartments with particles comprising associated therewith the second primers that also comprise one or more unique molecular identifier sequences (UMI) and one or more barcodes comprising known sequence that enables pooling of desired polynucleotides; releasing the primers from the beads; or (d2) exposing the hybrid molecules to a substrate comprising the second primers (that in some cases are region-specific with respect to spatial resolution of the subcellular structure) that also comprise one or more unique molecular identifier sequences (UMI) and one or more barcodes comprising known sequence that enables pooling of desired polynucleotides; release cDNA from the structure; (e) producing second strand synthesis upon hybridization of at least part of the second primer to the tail of the first complementary polynucleotides, thereby producing second complementary polynucleotides comprising at least part of the RNA sequence, the UMI, and the barcode; and (f) optionally amplifying the second complementary polynucleotide. In specific embodiments, a plurality of second complementary polynucleotides are amplified and/or sequenced. The second complementary polynucleotides may be amplified to produce amplified second complementary polynucleotides, followed by sequencing of one or more of the amplified second complementary polynucleotides. The amplifying may be by polymerase chain reaction or one or more isothermal amplification methods or linear amplification methods, including followed by sequencing of any kind, such as next-generation sequencing. The fixing step may comprise subjecting the cellular material to about 0.1% to 100% paraformaldehyde, and following the fixing step, the subcellular structures may be enriched, such as by flow cytometry or density gradient centrifugation. In some cases, following the fixing step, the subcellular structures are permeabilized, such as by one or more surfactants.

In particular embodiments, the subcellular compartment or structure can be a synaptosome, nucleus, organelle (mitochondria, ribosome, lysosome, endoplasmic reticulum, Golgi apparatus), polarized structures of the neurons (dendrites, axons, synapses, node of Ranvier, dendritic spine, axon initial segment), synaptic terminal, dendritic spines and cytoplasmic condensates; plastid, lysosome, or the physical structures that are secreted by a cell, such as extracellular vesicles.

In specific embodiments, the common tail sequence is a homopolymeric sequence, including one that was added to the 3′ end of the first complementary polynucleotides by terminal transferase. In some cases, the homopolymeric sequence comprises adenosines, and the second primers at least comprise thymosines. In particular aspects, the common tail sequence is added to the 3′ end of the first complementary polynucleotides by template switching activity of reverse transcriptase.

In particular embodiments, the microscopic volume is the scale of microliter, nanoliter, picoliter, or femtoliter volumes. The microscopic volume or microscope volume compartments may comprises droplets, including in some cases in microwells and microgels.

In some embodiments, the cDNA is released from the subcellular structures by a stimulus, such as a stimulus that comprises heating, pH changes, and/or enzymatic cleavage (RNAse H, RNase I, or both).

In specific embodiments, the second primers are attached to the beads by a linker or by a covalent bond. The primers may be released from the particles enzymatically, chemically (such as by ultraviolet radiation and/or a reducing agent), and/or physically (such as from heating).

In specific cases of the method, one or more of the first primers bind to intronic sequences in nascent RNA, or one or more of the first primers bind to long non-coding RNA. The long non-coding RNA may or may not comprise a polyadenylated tail.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIGS. 1a-1i. Overview of MATQ-Drop and the performance in species-mixing experiment. FIG. 1a, Reaction Scheme of MATQ-Drop. In situ reverse transcription and poly A tailing are performed on the fixed nuclei, which are then encapsulated in droplets with barcoded hydrogel beads. Inside the droplet, barcoded dT20 primers are enzymatically released from the beads to capture the poly A tail of cDNA released from the nuclei. After the barcoded second strand synthesis is accomplished, the emulsion is broken, and the product can be amplified and sequenced. FIG. 1b, Identification of the barcodes representing true nuclei in the species-mixing experiment. Barcodes are ordered from the largest to smallest UMI counts. On the UMI counts versus barcode rank plot, the knee point (162, red dashed line) indicates the threshold for true nuclei. FIG. 1c. Species annotation of the 162 nuclei identified. FIG. 1d, Species specificity of UMI. FIG. 1e, Fractions of UMI in exons and introns (Mean±SD). FIGS. 1f-1g. Detection sensitivity of MATQ-Drop in UMI counts and gene counts. FIGS. 1h-1i, Comparison of detection sensitivity between MATQ-Drop and other major single-nucleus RNA-seq methods^{8, 13} for single NIH/3T3 nuclei (p values calculated by two-sided t-test). Panel FIGS. 1d, 1f-1i, Boxplot: center line, median; box limits, upper and lower quartiles; whiskers, 1.5× interquartile range; points, outliers.

FIGS. 2a-2p. The human hippocampus synaptome atlas. FIG. 2a, Workflow of synapse preparation. FIG. 2b, FACS gating strategy to isolate the Hoechst-negative subneuronal structures (P8). FIG. 2c, Western blot showing the enrichment of synapse markers (Synaptophysin and Synapsin-1) and depletion of non-synapse markers (myelin: CNP; astrocytes: GFAP) in the sorted Hoechst-negative population (S: sorted, P: pellet before sorting. H: brain homogenate). FIG. 2d. UMAP visualization of synaptosome and neuron-glia junction subtypes of the human hippocampus. FIGS. 2e-2f, UMAP feature plots and violin plots showing the expression of subcellular-type-enriched markers in different clusters (HI-synapse: SYP; CA1 excitatory HI-synapse [Synapse_ExCA1]: FNDC1; CA3 excitatory HI-synapse [Synapse_ExCA3]: TSPAN18; DG excitatory HI-synapse [Synapse_ExDG]: SEMA5A; inhibitory HI-synapse [Synapse_In]: SLC6A1; LO-synapses: SHANK1, and SHANK3; ODC junctions: MBP, PLP1, and HIPK2; ASC junctions: AQP4, and GFAP). FIGS. 2g-2h, Detection sensitivity for each cluster in UMI counts and gene counts. FIG. 2i, Volcano plot showing the DEGs between hippocampus HI-synapses and LO-synapses. FIG. 2j, Pathway enrichment of hippocampus HI-synapse-enriched and LO-synapse-enriched genes. Fold enrichment is labeled next to the dots. FIG. 2k, Fraction of intronic UMI for each synaptosome and neuron-glia junction cluster in the human hippocampus. FIG. 2l, Volcano plot showing the DEGs between N-synapses and other synapses. in the hippocampus. FIG. 2m, Pathway enrichment of hippocampus N-synapse-enriched and other-synapse-enriched genes. Fold enrichment is labeled next to the dots. FIG. 2n. Volcano plot showing the differentially expressed genes between ODC-junctions and LO-synapses. FIG. 20, Volcano plot showing the differentially expressed genes between ASC-junctions and LO-synapses. FIG. 2p, Pathway enrichment of ODC junction-enriched and ASC junction-enriched genes. Fold enrichment is labeled next to the dots.

FIGS. 3a-3r. Nascent RNA-based cell atlas of the human hippocampus. FIG. 3a, Fractions of UMI in exons and introns. FIG. 3b, UMAP visualization of 11 cell populations identified in the primary clustering analysis. ExCA: CA excitatory neuron; ExDG: DG excitatory neuron; In_A-C: inhibitory neuron A-C; ASC1-2: astrocyte 1-2; OPC: oligodendrocyte precursor cell; ODC: oligodendrocyte; MG: microglia; T: T cells. FIGS. 3c-3d, Detection sensitivity for each hippocampus cluster in UMI counts and gene counts. FIG. 3e, UMAP feature plots showing the log normalized expression of the well-established cell-type-specific markers in different clusters (excitatory neuron: SLC17A7; CA neuron: FNDC1; DG neuron: SEMA5A; inhibitory neuron: GAD2; ASC: AQP4 and GFAP; OPC: CSPG4; ODC: MBP; MG: C3; T: CD96). FIG. 3f, Violin plots showing the marker gene expression level in different clusters. FIG. 3h, UMAP visualization of clustering results using only lncRNA expression matrix, colored by nascent RNA-based annotation. FIGS. 3g-3j. Volcano plots showing the exon-based DEGs between HI-synapses and nuclei for four neuronal subtypes. FIGS. 3k-3l, Venn diagram showing the overlap of synapse-enriched or nucleus-enriched genes among four neuronal subtypes. FIG. 3m, Pathway enrichment of shared synapse-enriched and nucleus-enriched genes. Fold enrichment is labeled next to the dots. FIG. 3n. The average intronic UMI fraction in HI-synapses versus nuclei of CA excitatory neurons, with the marginal rug plot indicating density. FIG. 3o, Identification of the unspliced synaptic genes in CA excitatory neurons. FIG. 3p, Number of the unspliced synaptic genes grouped by gene type. FIG. 3q. Venn diagram of the unspliced synaptic genes across four neuronal subtypes and the list of 41 protein-coding genes detected in all four neuronal subtypes. FIG. 3r, Pathways enriched in fully spliced genes, identified through preranked GSEA based on splicing score.

FIGS. 4a-4j. The mouse hippocampus cell atlas and synaptome atlas. FIG. 4a, UMAP visualization of synaptosome and neuron-glia junction subtypes of the mouse hippocampus. FIG. 4b, Violin plots showing the expression of subcellular-type-enriched markers in different clusters (Syn1: Csmd1, Kcnip4, and Nrg3; Syn2: Grin2b; Syn3: Shank1, and Camk2a; Syn4: Pclo; Syn5: Zbtb20; Syn6: Chd9; Syn7: Purg; Syn8: Nopchap1; Syn9: Apc; Syn10: Hivep3; Syn11: Kmt2d; Syn12: Ksr; AIS/NR: Ank3, and Ank2; ODC junctions: Mbp, and Mobp; ASC junctions: Slc1a2, and Atp1a2). FIG. 4c, Fraction of intronic UMI for each synaptosome and neuron-glia junction cluster in the mouse hippocampus. FIG. 4d, UMAP visualization of 19 cell populations identified in the primary clustering analysis. ExCA1A-B: CA1 excitatory neuron A-B; ExCA3: CA3 excitatory neuron; ExDG: DG excitatory neuron; ExSub: subiculum excitatory neuron; Ex1-4: other excitatory neuron 1-4; In1-2: inhibitory neuron 1-2; ASC1-2: astrocyte 1-2; OPC: oligodendrocyte precursor cell; ODC: oligodendrocyte; MG1-3: microglia 1-3; Fibroblast: Fibroblast. FIG. 4e. Volcano plots showing the exon-based DEGs between synapses and neuronal nuclei. FIG. 4f, Pathways enriched in the synapses and nuclei, identified through GSEA. FIG. 4g. The average intronic UMI fraction in synapses versus neuronal nuclei, with the marginal rug plot indicating density. FIG. 4h, Identification of the unspliced synaptic genes in neurons. FIG. 4i, Number of the unspliced synaptic genes grouped by gene type. FIG. 4j, Pathways enriched in unspliced and fully spliced genes, identified through preranked GSEA based on splicing score.

FIGS. 5a-5f. Alzheimer's Disease-associated synaptopathy. FIG. 5a. The comparison of cell frequency for ODG and MG1 cells between 5×FAD mice and WT. T-test significance code: p values (0,0.0001]: ****; (0.0001,0.001]: ***; (0.001,0.01]: **; (0.01,0.05]: *. FIG. 5b, Functional enrichment of the DEGs between the single-nucleus transcriptome of 5×FAD and WT for different cell types. FIG. 5c, Heatmap showing the fold change of intron-based DEGs (abs(log₂FC)>log₂ (1.3), FDR<0.05) between 5×FAD and WT in different types of nuclei. FIG. 5d, Heatmap showing the fold change of DEGs (abs(log₂FC)>log₂ (1.3), FDR<0.05) between 5×FAD and WT in different synaptosome and neuron-glia junction subtypes. FIG. 5e, Pathways enriched in 5×FAD synapses compared to WT. FIG. 5f. Heatmap showing the fold change of synapse-AD-DEGs shared by at least 6 subtypes, and their intron-based expression fold changes in the AD nuclei.

FIGS. 6a-6l. The cell typing using only lncRNA species and the detection sensitivity comparison between 10× Chromium and MATQ-Drop.

FIG. 6a, UMAP visualization of clustering results using only lncRNA expression matrix from the single-nucleus transcriptome of the human hippocampus. FIG. 6b, Heatmap showing the scaled expression levels of cell-type-specific lncRNAs. FIG. 6c, UMAP feature plot showing the log normalized expression level of cell-type-specific lncRNAs (excitatory neuron: LY86-AS1; inhibitory neuron: DLX6-AS1; ASC: PPP1R9A-AS1; OPC: AC124254.2; ODC: LINC01608; MG: LINC01141). FIG. 6d, UMAP visualization of clustering results using only lncRNA expression matrix from the single-nucleus transcriptome of mouse hippocampus. FIGS. 6c-6e, UMAP feature plot showing the log normalized expression level of cell-type-specific lncRNAs (CA1 excitatory neuron: 4921539H07Rik; CA3 excitatory neuron: Gm32647; DG excitatory neuron: Gm12339; subiculum excitatory neuron: Gm28905; inhibitory neuron: Gm45323, and Dlx6os1; ASC: Celrr; OPC: 6030407003Rik; ODC: Gm16168; MG: AU020206; Fibroblast: 2610307P16Rik). FIGS. 6f-6g. Transcript-based detection sensitivity (UMI count and gene count) compared to 10× Chromium³⁹. FIGS. 6h-6i, Exon-based detection sensitivity (UMI count and gene count) compared to 10× Chromium³⁹. FIG. 6j. LncRNA detection sensitivity (UMI count) compared to 10× Chromium³⁹. FIG. 6k, Accumulated fraction of UMI on the axis of the ranked lncRNA genes. FIG. 6l, LncRNA detection sensitivity (gene count) compared to 10× Chromium³⁹. T-test significance code: p values (0,0.0001]: ****; (0.0001,0.001]: ***; (0.001,0.01]: **; (0.01,0.05]: *.

### DETAILED DESCRIPTION

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z.” “x or (y and z),” or “x or y or z.” It is specifically contemplated that x, y, or z may be specifically excluded from an embodiment.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “semi-amplicon” refers to polynucleotides that are products after reverse transcription, such as cDNA. As used herein, the term “full amplicon” refers to polynucleotides that are a second strand synthesis product or are amplified molecules from full amplicons. Amplicons have common adapters on both ends, which allow further amplification, including for PCR amplification. Amplicons may be present in a library with other amplicons, the combination of which may represent a desired set of RNA templates, such as RNA in or associated with a substructure.

The term “barcode” can refer to a known polynucleotide sequence that allows some feature of a polynucleotide with which the barcode is associated to be identified. In some cases, the feature of the polynucleotide to be identified is the sample from which the polynucleotide is derived. In some cases, barcodes are at least 3, 4, 5, 6, 7, 8, 9, 10, 11, 12, 13, 14, 15, or more nucleotides in length. In some cases, barcodes are shorter than 10, 9, 8, 7, 6, 5, or 4 nucleotides in length. An oligonucleotide (e.g., primer or adapter) can comprise about, more than, less than, exactly, or at least 1, 2, 3, 4, 5, 6, 7, 8, 9, or 10 different barcodes. In some cases, barcodes associated with some polynucleotides are of different length than barcodes associated with other polynucleotides. Barcodes can be of sufficient length and comprise sequences that can be sufficiently different to allow the identification of samples based on barcodes with which they are associated. In some cases, each barcode in a plurality of barcodes differ from every other barcode in the plurality at one or more nucleotide positions, such as (in some cases, at least) 1, 2, 3, 4, 5, 6, 7, 8, 9, 10, or more positions. In some cases, an adapter comprises at least one of a plurality of barcode sequences. In some cases, barcodes for a second adapter oligonucleotide are selected independently from barcodes for a first adapter oligonucleotide. In some cases, first adapter oligonucleotides and second adapter oligonucleotides having barcodes are paired, such that adapters of the pair comprise the same or different one or more barcodes. In some cases, the methods described herein further comprise identifying the sample from which a target polynucleotide is derived based on a barcode sequence to which the target polynucleotide is joined. A barcode can comprise a polynucleotide sequence that when joined to a target polynucleotide serves as an identifier of the sample from which the target polynucleotide was derived.

The term “cellular material” as used herein refers to whole cells or parts of cells, including cell fragments. When the cellular material comprises material that is less than a whole cell, the parts of the cells may or may not be naturally derived. In specific cases, the cellular material comprises whole subcellular structures and/or parts of subcellular structures.

The term “long non-coding RNA (or lncRNA)” as used herein refers to RNA transcripts having lengths greater than about 200 nucleotides that are not translated into protein.

The term “nascent RNA” as used herein refers to RNA synthesized by RNA polymerase II prior to post-transcriptional processing (such as capping, tailing, and splicing) or prior to completion of post-transcriptional processing.

The term “subcellular structure” as used herein refers to one or more physical structures within a cell, such as the nucleus, organelles (mitochondria, ribosome, lysosome, endoplasmic reticulum, Golgi apparatus), polarized structures of the neurons (dendrites, axons, synapses, node of Ranvier, dendritic spine, axon initial segment); synaptic terminal, dendritic spines and cytoplasmic condensates; or the physical structures that are secreted by a cell, such as extracellular vesicles.

The present disclosure concerns methods of amplifying RNA sequences, as representative sequences in a DNA library, when the corresponding RNA molecules are a part of or otherwise associated with subcellular structures. According to certain aspects of the disclosure, RNA molecules of single subcellular particles are stably attached to subcellular structures, such as by fixation. The solid subcellular body may include any part of the original cell, the whole or the part of the original nucleus, a cellular or subcellular structure, and synthesized particles, such as polymeric gel beads. Herein they may be referred to as microscopic biological particles. The attachment of RNA to the solid particle can be achieved physically or chemically. In some instances, the RNA can be attached to the solid particle by covalent bonds, by hydrogen bonds, by protein-protein interaction, or by magnetic force.

In situ reverse transcription is performed by at least one reverse transcriptase to synthesize the cDNA based on RNA attached to microscopic biological particles. The hybridization between the cDNA and the RNA template allows the indirect but stable attachment of the cDNA to the subcellular-specific biological particles. A common sequence is then added to the 3′ end of the cDNA in situ. In some instances, the common sequence is a homopolymer, which can be added by a terminal transferase. In some instances, the common sequence can be added by a reverse transcriptase with template-switching activity. The cDNA with a common sequence on the 3′ end can be amplified with at least one DNA polymerase. Details of this and subsequent second strand synthesis to produce the library are provided herein.

To meet specific technical demands for high-throughput transcriptome profiling method of individual subcellular structures, the disclosure provides the development of a microvolume-based total-RNA scRNA/sssRNA seq platform, referred to as Multiple-Annealing-and-Tailing-based Quantitative RNA-seq in Droplet, or MATQ-Drop. The development of MATQ-Drop is based on the previous chemistry of MATQ-seq⁶. MATQ-Drop works with fixed samples, and its effective detection of nascent RNA makes it suitable for characterizing local splicing in synaptosomes (as an example of subcellular structure). While the commercial 10× Genomics Chromium platform is broadly accessible⁷⁻¹⁰. SMART-seq based chemistry¹¹ on this platform is mainly designed for quantifying mature RNA levels in fresh and nonfixed samples, hence, making it unsuitable for transcriptome profiling of single-subcellular compartment, such as synaptosomes.

Using the MATQ-Drop platform, the inventors performed the transcriptome profiling of single synaptosomes of human and mouse brain samples. For convenience, the transcriptome of synaptosomes is referred to as synaptome. In the synaptome data, the inventors were able to identify various types of neurites, including different subtypes of synaptosomes and neuron-glia junctions. Among different subtypes of synaptosomes, presynaptic and postsynaptic clusters were observed, as well as a special subcluster associated with the synapses in the process of assembly and maturation. Transcriptomic differences between different subclusters can be readily detected. With the effective detection of nascent RNAs, the landscape of intron-retention was characterized for various clusters of synapses.

In addition to synaptome profiling, MATQ-Drop was applied to profile the transcriptome of single nuclei for the same brain samples. With both synaptome and the single-nucleus transcriptome, the inventors were able to connect subclusters of synapses to different types of neurons. The differential gene expression and splicing between the synapses and neuronal nuclei was then analyzed. Furthermore, the synaptosomes isolated from an Alzheimer's disease (AD) mouse model were profiled, and the synaptopathy-associated transcriptome was characterized, leading to discovery of the novel AD-associated gene expression changes that cannot be detected by single-nucleus transcriptome profiling.

With the effective detection of total RNA, the inventors also successfully generated the cell atlas using only long non-coding RNA (lncRNA) species. This result indicates that MATQ-Drop allows the large-scale identification of the cell type-specific lncRNA species. Furthermore, based on the single nucleus transcriptome of the mouse hippocampus, the inventors also conducted a benchmark comparison between MATQ-Drop and 10× Chromium. The result shows that MATQ-Drop demonstrated a 2.5-3.7 fold improvement of gene detection sensitivity compared to the 10× platform. Overall, as the first total-RNA based high-throughput transcriptome platform, MATQ-Drop provides an alternative high-throughput high-sensitivity SC transcriptome platform to the 10× Chromium platform. The transcriptome profiling of individual synaptosomes based on MATQ-Drop facilitates new discoveries in neurosciences.

In specific embodiments, the disclosure concerns microvolume-based high-throughput transcriptome profiling of individual synapses using total-RNA-Seq chemistry.

### I. General Embodiments

Embodiments of the disclosure allow for producing libraries that represent RNA molecules of any kind, including at least for mRNA molecules, nascent RNAs, microRNAs, and long non-coding RNAs, for example. In some cases, the RNAs represent RNA in (or otherwise associated with) a cellular substructure. As a result of producing libraries from multiple types of RNA, including other than only mRNA, one can obtain, for example, gene expression information based on unspliced transcript sequences. This may result when sequencing the produced library molecules at least some of which may map to intronic regions, as opposed to sequencing of sequence from spliced transcripts representing only exonic regions.

Embodiments of the disclosure also include methods for identifying different subtypes of subcellular structures, where applicable. Given that the disclosed methods provide for detection of at least nascent RNAs, one can apply the methods to profile splicing at temporal and/or spatial levels. For example, one can apply the methods to analyze (even at a large-scale level) multiple subcellular structures from one (or more) samples that can demonstrate whether or not there are transcriptomic differences among the subcellular structures, such as from a similar region. For example, the methods may detect differences in the subcellular structure RNAs in a gradient fashion or having regional differences in a particular area being analyzed. In specific cases, one can seek or identify whether there are specific types of subclusters of cells based on clustering of subcellular structure transcriptomes.

Embodiments of the disclosure include methods that utilize a series of steps to produce a library of amplicons that represent template RNA of any kind, including polyadenylated and/or non-polyadenylated RNA, and not necessarily only mRNA transcripts. Generally speaking, RNA from cellular material of any kind comprising subcellular structures of any kind is utilized as a template to produce amplicons representative of the RNA, and the amplicons can be sequenced or processed in any manner. The methods in particular embodiments concern fixing cellular material for which RNA is in or is associated with subcellular structures of any kind. The RNA of the fixed subcellular structure/RNA complexes are exposed to sufficient in situ reverse transcription conditions (and using specific types of primers) followed by in situ tailing of the 3′ ends of the newly synthesized complementary polynucleotide molecules to the RNA. In cases wherein the template RNA is RNA, the newly synthesized complementary polynucleotide molecules may be referred to as cDNAs. In cases wherein the template RNA is nascent RNA, microRNA, or long non-coding RNA, the newly synthesized complementary polynucleotide molecules may also be referred to as complementary DNA, or in some cases semi-amplicons. The tailing of the 3′ end of the newly synthesized complementary polynucleotide molecules allows for a common sequence among the newly synthesized complementary polynucleotide molecules by which a primer can bind for second strand synthesis, subsequently allowing at least for further linear amplification of the original RNA template sequence. In cases wherein grouping of the specific newly synthesized complementary polynucleotide molecules is desired, the primers utilized for second strand synthesis may comprise one or more barcodes. In cases where particular identification of the specific newly synthesized complementary polynucleotide molecules is desired, the primers utilized for second strand synthesis may comprise one or more unique molecular identifier sequences unique to each polynucleotide molecule. At least one result of the method produces amplicons that comprise sequence representing at least part of the original RNA template (including representing intronic and other non-coding sequences, in at least some cases) and a barcode and, in at least some cases, the unique molecular identifier.

In an initial step of the method, cellular material is obtained, such as commercially or from a biological or clinical sample from one or more individuals. The source of the material may be fresh, frozen, or it was previously frozen. The cellular material may comprise whole cells or fragments of cells and comprises subcellular structures of any kind. In specific embodiments, the subcellular structure is a synaptosome, a nuclei, a plastid, or a mitochondria. The cellular material/RNA is histologically fixed under suitable physical and/or chemical conditions such that the RNA is physically and/or chemically linked to the cellular material, including the subcellular structures. In specific embodiments, the cellular material/RNA is fixed by one or more crosslinking fixative compounds, such as that generate covalent chemical bonds between the RNA and the cellular material, including the subcellular structures. The fixative may be one or more aldehydes, such as paraformaldehyde, formaldehyde, glutaraldehyde, or a combination thereof; one or more alcohols, such as protein-denaturing methanol, ethanol and/or acetone; one or more oxidizing agents, such as osmium tetroxide, potassium dichromate, chromic acid, and/or potassium permanganate; one or more zinc fixatives, such as zine acetate and/or zinc chloride; or a combination thereof. In specific examples, the fixative is in the range of about 0.1-100, 0.1-50, 0.1-25, 0.1-10, 0.1-5, 0.1-1, 1-100, 1-50, 1-25, 1-10, 1-5, 5-100, 5-50, 5-25, 5-10, 10-100, 10-50, 10-25, 25-100, 25-50, or 50-100%.

Following fixation, the cellular material/RNA complexes are subjected to appropriate in situ reverse transcription conditions that utilize certain primers. Embodiments of the methods utilize primers that facilitate production of complementary polynucleotides upon the binding and extension by the primers. In specific cases the complementary polynucleotides are produced upon in situ reverse transcription in which the RNA is exposed to at least one reverse transcriptase in the presence of a sufficient amount of primers that comprise random sequence and that can bind the RNA. The primers in specific embodiments comprise random sequence that allows for them to bind anywhere upon a mRNA, nascent RNA, or long non-coding RNA. The primers allow for production of double-stranded complementary DNA (cDNA) from the total RNA of one or more cells. Double-stranded cDNA produced according to the disclosed amplification method is suitable for further amplification, whether or not by nonlinear means.

In a particular embodiment, there is annealing of multiple primers to the same RNA molecule. Upon exposure of the primers to the nucleic acid, this generates a mixture comprising primer-annealed nucleic acid templates. In specific embodiments, production of complementary polynucleotides to the template RNA molecules (mRNA, nascent RNA, long non-coding RNA) utilizes a shotgun coverage approach in which multiple primers will hybridize on a single RNA template molecule, and this will occur across a plurality of RNA molecules, regardless of whether the RNA is polyadenylated or non-polyadenylated. The primers in totality can hybridize to introns, exons, 5′ ends of RNAs, and 3′ ends of RNAs, although a specific primer may be able to hybridize to both an intron and an exon, such as across a splice junction. Therefore, a combination of primers that initiate reverse transcription can cover a single RNA molecule, and that combination may include 2, 3, 4, 5, or more primers (although in alternative embodiments only one primer binds a particular RNA molecule). In specific embodiments, the sequence design of the primers allows them to hybridize to RNA transcripts at low temperature without hybridizing to each other to avoid the production of the primer dimers.

In specific embodiments, the primers in the first plurality are about 40%-60% G-rich or about 40%-60% C-rich, although not simultaneously. In specific embodiments, the primers comprise the following formula: 5′-XnYmZp-3′, wherein n is greater than 2 (or greater than 4, 6, 8, 10, 12, 14, 16, 18, 20, 22, 24, 26, 28, 30, 32, 33, 34, or 35) and X is or is about 40%-60% G-rich (including about 40%-60%, 40%-55%, 40%-50%, 40%-45%, 45%-60%, 45%-55%, 45%-50%, 50%-60%, 50%-55%, 55%-60%) or is or is about 40%-60% C-rich (including about 40%-60%, 40%-55%, 40%-50%, 40%-45%, 45%-60%, 45%-55%, 45%-50%, 50%-60%, 50%-55%, 55%-60%), wherein Y is any nucleotide and m is 5-8 nucleotides (including 5-8, 5-7, 5-6, 6-8, 6-7, or 7-8 nucleotides, and including 5, 6, 7, or 8 nucleotides) and wherein Z is a T or a G when X is G-rich, or Z is a C when X is C-rich, wherein p is about 2-20 (including 2-20, 2-28, 2-26, 2-14, 2-12, 2-10, 2-8, 2-6, 2-4, 4-40, 4-18, 4-16, 4-14, 4-12, 4-10, 4-8, 4-6, 6-20, 6-18, 6-16, 6-14, 6-12, 6-10, 6-8, 8-20, 8-18, 8-16, 8-14, 8-12, 8-10, 10-20, 10-18, 10-16, 10-14, 10-12, 12-20, 12-18, 12-16, 12-14, 14-20, 14-18, 14-16, 16-20, 16-18, or 18-20) nucleotides. In specific embodiments, n is about 20-35, 20-32, 20-30, 20-28, 20-26, 20-25, 20-24, 20-22, 22-35, 22-34, 22-32, 22-30, 22-28, 22-26, 22-25, 22-24, 24-35, 24-34, 24-32, 24-30, 24-28, 24-26, 24-25, 25-35, 25-34, 25-32, 25-30, 25-28, 25-26, 26-35, 26-34, 26-32, 26-30, 26-28, 28-35, 28-34, 28-32, 28-30, 30-35, 30-34, 30-32, 32-35, 32-34, or 34-35 nucleotides. In some cases, n may be about 20, 21, 22, 23, 24, 25, 26, 27, 28, 29, 30, 31, 32, 33, 34, or 35 nucleotides in length. In specific cases, the plurality of primers are designed to avoid crosstalk among them.

In specific cases for the primers, the formula of 5′-XnYmZp-3′ may be 5′ DnYmZp 3′ or 5′-HnYmZp-3′, wherein D represents G or A or T, and H represents C or A or T. In specific cases, n is between about 20 to about 35 nucleotides, including about 20-35, 20-32, 20-30, 20-28, 20-26, 20-25, 20-24, 20-22, 22-35, 22-34, 22-32, 22-30, 22-28, 22-26, 22-25, 22-24, 24-35, 24-34, 24-32, 24-30, 24-28, 24-26, 24-25, 25-35, 25-34, 25-32, 25-30, 25-28, 25-26, 26-35, 26-34, 26-32, 26-30, 26-28, 28-35, 28-34, 28-32, 28-30, 30-35, 30-34, 30-32, 32-35, 32-34, or 34-35 nucleotides. In some cases, n may be 20, 21, 22, 23, 24, 25, 26, 27, 28, 29, 30, 31, 32, 33, 34, or 35 nucleotides in length.

In specific embodiments, for Xn, Hn, or Dn, the respective G or C bases are well dispersed in the X sequence, including to avoid any clustering of the same base. In specific cases, a G or C is separated by 3, 4, 5, 6, or more bases.

According to one aspect, the reaction mixture to produce the complementary polynucleotides in in situ reverse transcription is subjected to conditions that promote primer-template annealing. In at least some cases, this involves lowering the temperature of the mixture to a temperature that allows random nucleotides at the 3′ end of the primer to anneal to the RNA to form hybrid duplexes. In specific cases, the temperature may be as low as 0° C. and may be as high as about 60° C. Thus, in specific embodiments the temperature for in situ reverse transcription is about 0-60, 0-50, 0-40, 0-30, 0-20, 0-10, 10-60, 10-50, 10-40, 10-30, 10-20, 20-60, 20-50, 20-40, 20-30, 30-60, 30-50, 30-40, 40-60, 40-50, or 50-60° C.

After the hybrid duplexes form, one or more reverse transcriptases present in the reaction mixture extends the cDNA strand from the 3′ end of the first primer during an appropriate incubation period and to produce hybrid molecules between the RNA and cDNA. The process of hybrid duplex formation and cDNA extension is repeated at least 2 times, although it may occur 3, 4, 5, 6, 7, 8, 9, 10, 11, 12, 13, 14, 15, 16, 17, 18, 19, or 20 or more times. In the repetition of this step, there is no subjecting of the reaction to melting temperatures. In at least some cases, following first-strand cDNA synthesis by reverse transcriptase, the reaction mixture may be subjected to conditions wherein unannealed primers and template RNA are digested and enzymes present in the reaction are made inactive. In particular cases, the primers are digested prior to digestion of the template RNA. The digestion of the primers may occur by any manner, but in specific embodiments it occurs with a nuclease. In embodiments of the disclosure, methods are provided that can efficiently remove preexisting primers to allow efficient tailing of the first-strand cDNAs. Without efficient digestion of primers, the tailing of residual primers out-competes the tailing of semi amplicons and leads to the failure of amplification in the following step. Thus, in certain aspects one can use T4 DNA polymerase or other polymerases with exonuclease activities at low temperature below (30° C. or below) and Exonuclease I or other exonucleases that only digest unannealed primers. The enzymes can be heat-inactivated.

Following production of the polynucleotides that are complementary to the RNA, the mixture may be subject to in situ tailing. The 3′ ends of the complementary polynucleotides may be tailed with a sequence that is known and common among the complementary polynucleotides and that is complementary to primers utilized for second strand synthesis and further linear amplification (and which are barcoded, in specific embodiments). In specific embodiments, the tailing step occurs at a range of temperature of about 10-45° C., such as about 10-45, 10-40, 10-35, 10-30, 10-25, 10-20, 10-15, 15-45, 15-40, 15-35, 15-30, 15-25, 15-20, 20-45, 20-40, 20-35, 20-30, 20-25, 25-45, 25-40, 25-35, 25-30, 30-45, 30-40, 30-35, 35-45, 35-40, or 40-45° C. The tailing of the 3′ end may occur by any method, but in specific embodiments it occurs by terminal transferase. The tailing may be homopolymeric with a single nucleotide and in specific embodiments the polynucleotide is an A. T. C or a G, but in specific cases it is an A. That is, in specific embodiments, 3′ end tailing can be conducted with concentrated A base in the presence of terminal deoxynucleotidyl transferase, wherein the base used for tailing will be complementary to the barcode primers. The length of the tail may be of any length but in particular may be in the range of 1-3000, 1-2000, 1-1000, 1-500, 1-100, 100-3000, 100-2000, 100-1000, 100-500, 500-3000, 500-2000, 500-1000, 1000-3000, 1000-2000, or 2000-3000 bases.

In other embodiments, the 3′ ends of the complementary polynucleotides may be tailed with a sequence that is known and common among the complementary polynucleotides, but the method utilizes the template switching activity of reverse transcriptase instead of using terminal transferase. In this example, one can utilize suitable levels of one or more template switching oligonucleotides with reverse transcriptase and the subcellular structures/hybrid molecules between the RNA and cDNA.

In particular embodiments of the method, the tailed complementary polynucleotides are further amplified linearly using primers that allow recognition of certain subgroups, and at least in some cases this generates a library for subsequent nonlinear amplification of some or all of the library for further analysis. In specific embodiments, at least the second strand synthesis step occurs in the scale of a microscopic volume microliter, nanoliter, picoliter, or femtoliter volumes and in some cases no greater than microliter, nanoliter, picoliter, or femtoliter volumes. In specific cases, the microscopic volume is within a compartment or substrate, although in alternative cases it is not in a compartment. In certain aspects, the microscopic volume or microscope volume compartments comprises droplets, and the droplets may be in microwells, or oil or chip devices, such as microwells on polydimethylsiloxane (PDMS) or glass materials.

In specific embodiments, the RNA/cDNA hybrid as part of a fixed complex with the subcellular structures is encapsulated in a microscopic volume or microscope volume compartments. The microscopic volume or microscope volume compartments may or may not already comprise particles (such as beads, including gel beads) that have associated therewith (such as attached by a linker or through a deoxyUridine) the barcode primers. In certain aspects of the method, the primer-linked beads are generated, such as following design of the barcode primers. The primer-linked beads may be generated by the user or obtained chemically, in some cases.

In specific embodiments, certain steps of the method may be practiced in the following example of an order: (1) production of cDNA in which the RNA is still fixed to the subcellular structure; (2) encapsulation of the RNA/cDNA hybrid with the subcellular structure in the droplet with the particles (e.g., beads); (3) release of barcoded primers from the beads; (4) release of cDNA from the subcellular structure; and (5) production of the amplicons from the cDNA using the barcoded primers (i.e., second strand synthesis).

Following droplet encapsulation, the cDNA is released from the subcellular structures by a stimulus, such as a stimulus comprises heating, pH changes, and/or enzymatic cleavage (RNAse H, RNase I, or both). The droplet comprises suitable reagent(s) to allow hybridization of the tail of the tailed cDNA to the primer and second strand synthesis (discussed below).

Prior to second strand synthesis, the primers may be released from the particles (such as the beads) by physical or chemical means. The primer may be released by enzymatic means. When the primer is attached to the particle through a deoxyUridine, the primer may be released by a mixture of Uracil DNA glycosylase (UDG) and the DNA glycosylase-lyase Endonuclease VIII (e.g., the USER™ enzyme).

In alternative cases, instead of the second strand synthesis occurring associated with a barcode primer-linked particle (such as a bead), the RNA/cDNA hybrid molecules are exposed to a substrate comprising the barcode primers.

For second strand synthesis, the reaction mixture is exposed to at least one DNA polymerase and a plurality of barcode primers that comprise a barcode having known sequence and that enables grouping of particular polynucleotides with the same or similar (>about 90%, 91%, 92%, 93%, 94%, 95%, 96%, 97%, 98%, 99%, or greater in sequence identity) barcode. In specific cases, the barcode primers may have the XnYmZpTq sequence motif, wherein n is greater than 2 and X is about 40%-60% G-rich or about 40%-60% C-rich, Y is the DNA barcode sequences as the unique sample indexes (a specific DNA sequence of 5, 6, 7, or 8 bases, m are in range of 5-8) and Z is the random sequence of 5N, or 6N, or 7N or 8N (p are in range of 5-8) as the unique indexes of single molecules; T is the thymine base with q value range from 16 to 32 to capture the polyA tail of cDNA. In any case, the barcode primers are designed to avoid crosstalk among them and avoid primer dimers. In specific embodiments, the generation of secondary cDNA occurs at a temperature in the range of about 42-72° C., such as about 42-72, 42-65, 42-60, 42-55, 42-40, 42-45, 45-72, 45-65, 45-60, 45-55, 45-50, 48-72, 48-65, 48-60, 48-55, 48-50, 50-72, 50-65, 50-55, 55-72, 55-65, 55-60, 60-72, 60-65, or 65-72° C.

The reaction mixture is subjected to conditions that promote hybridization between the barcode primer and the cDNA molecule that was produced upon in situ reverse transcription and followed by tailing. After the hybrids form, one or more polymerases present in the reaction mixture extend the second cDNA strand from the 3′ end of the first primer during an incubation period. The produced molecules comprise sequence representative of the original RNA template and barcode sequence. Multiple second strand syntheses of the tailed complementary polynucleotide for the original template RNA may occur, and multiple complementary strand synthesis may occur of these generated second strands, and so on. Therefore, in particular embodiments, the methods produce second strand synthesis upon hybridization of at least part of the barcode primer to the tail of the RNA-complementary polynucleotides, thereby producing polynucleotides comprising at least part of the template RNA sequence, the UMI, and the barcode. After the second synthesis is completed, the droplet may be broken to release the library amplicon molecules. In some cases, part or all of the library is stored, and in other cases part or all of the library is utilized, such as by amplifying, optionally followed by sequencing. The library may be stored after amplification.

In specific embodiments, the produced molecules are a library representing template RNA molecules. The library may be configured for commercial or research use, in some cases. In specific embodiments, part or all of the library may be amplified by suitable methods. In cases wherein only part of the library is amplified, the part may or may not include a pooling of polynucleotides that comprise one or more certain barcodes, such as to the exclusion of polynucleotides that lack these one or more certain barcodes. Polynucleotides with certain UMIs may be amplified.

The amplification of library molecules may be by any suitable method, including by thermal amplification methods or isothermal amplification methods. The library molecules may be sequenced subsequent to amplification and/or prior to amplification. In specific embodiments, the amplification is by polymerase chain reaction, and the amplified molecules may be sequenced by next-generation sequencing or other sequencing platforms.

In specific embodiments, after the second synthesis is completed, the droplet may be broken and the PCR reaction may be performed, such as to amplify the library for next-generation sequencing.

### II. Barcodes

PCR amplification bias is a significant challenge in RNA sequencing as small differences in amplification efficiency can lead to significant artificial signals in the data. To address this issue, one can introduce random “barcodes” (random DNA sequence with variable length (for example NNNNNN, where N represents one of the four standard nucleotides, in specific embodiments) into the primers, which will index each unique produced double-stranded cDNA product. Following sequencing, and in one example, by indexing each of the reads with barcodes, one can differentiate high copy genes (highly expressed genes) from amplicons with high amplification efficiency (e.g. a high copy gene with many unique barcodes compared to a high copy gene with only one barcode). Such an application significantly improves the accuracy of sequencing data and captures biologically meaningful information, such as gene expression analysis and characterization of substructures. The disclosed methods provide a solution to normalize gene expression from sequencing data using the barcodes.

### III. Exemplary Applications of Methods of the Disclosure

Methods of the disclosure may be utilized in research, clinical, and/or other applications. In particular embodiments, methods of the disclosure are utilized in diagnostics and/or prognostics and/or monitoring of one or more therapies for an individual, for example. In some cases, the party preparing the library may or may not be the party or parties performing the amplification of the library and also may or may not be the party or parties performing analysis of the library, whether amplified or not. A party applying information from the analysis of the amplified library may or may not be the same party that performed the method of preparing the library and/or amplifying part or all of it.

In one example of an application of one or more methods of the disclosure, the method is utilized for assaying for one or more variations in content or expression level of one or more nucleic acids related to substructures from an individual; the variation may or may not be in relation to a known standard, for example, such as a corresponding wild-type sequence of a particular nucleic acid of a substructure. The variation in content may comprise one or more nucleotide differences compared to wild-type, such as a substitution, deletion, inversion, and so forth. The variation in expression may comprise upregulation or downregulation compared to normal expression levels of a particular known or determined standard. The standard may comprise the content of normal nucleic acid content or expression level in cells known to be normal in genotype and/or phenotype.

In specific embodiments, one or more of the amplified library amplicons is analyzed for one or more of substructure-related genes (such as identifying markers), cancer mutations, gene fusion products, splice variants, the expression of oncogenes, the loss of expression of tumor suppressors, the expression of tumor-specific antigens, and/or the expression of all the expressed genes.

In specific cases, the nucleic acid being assayed for is obtained from a sample from an individual that has a medical condition or is suspected of having a medical condition or is at risk for having a medical condition or is undergoing therapy for a medical condition. The sample may be of any kind so long as nucleic acid may be obtained directly or indirectly from one or more cells from the sample, and the nucleic acid may be indicative of the presence or type of a cellular substructure. In particular embodiments, the nucleic acid is obtained from one or more cells from a sample from the individual. The sample may be blood, tissue, hair, biopsy, urine, nipple aspirate, amniotic fluid, cheek scrapings, fecal matter, or embryos.

An appropriate sample from the individual is obtained, and the methods of the disclosure may be performed directly or indirectly by the individual that obtained the sample or the methods may be performed by another party or parties.

In some cases, in order to obtain sufficient nucleic acid for testing, a blood volume of at least 1, 2, 3, 4, 5, 10, 20, 25, 30, 35, 40, 45, or 50 mL is drawn. In some cases, the starting material is peripheral blood. The peripheral blood cells can be enriched for a particular cell type (e.g., mononuclear cells; red blood cells; CD4+ cells; CD8+ cells; immune cells; T cells, NK cells, or the like). The peripheral blood cells can also be selectively depleted of a particular cell type (e.g., mononuclear cells; red blood cells; CD4+ cells; CD8+ cells; immune cells; T cells, NK cells, or the like).

In particular embodiments the starting material comprises cellular material that comprises subcellular structures for which RNA analysis is specifically intended. In some cases, the starting material can be a tissue sample (and may be a biopsy) comprising a solid tissue, with non-limiting examples including brain, neuronal, liver, lung, kidney, prostate, ovary, spleen, lymph node (including tonsil), thyroid, pancreas, heart, skeletal muscle, intestine, larynx, esophagus, and stomach. In other cases, the starting material can be cells containing nucleic acids, immune cells, and in particular immune cells. In some cases, the starting material can be a sample containing nucleic acids, from any organism, from which genetic material can be obtained. In some cases, a sample is a fluid, e.g., blood, saliva, lymph, or urine.

In some cases, a sample can be taken from a subject with a condition. In some cases, the subject from whom a sample is taken can be a patient, for example, a patient with neurodegenerative disease (or suspected thereof), or a cancer patient or a patient suspected of having cancer. The subject can be a mammal, e.g., a human, and can be any gender. In some cases, the subject is a female and is pregnant. In some cases, the subject can be receiving therapy for treatment of a condition. In some cases, the therapy can be for treating cancer. In some cases, the therapy can be immunotherapy. The sample can be a tumor biopsy. The biopsy can be performed by, for example, a health care provider, including a physician, physician assistant, nurse, veterinarian, dentist, chiropractor, paramedic, dermatologist, oncologist, gastroenterologist, or surgeon.

In particular applications, one or more particular nucleic acid sequences are desired to be known in a sample from an individual. The individual may be of any age. The individual may be subjected to routine testing or may have a particular desire or medical reason for being tested. The individual may be suspected of having a particular medical condition, such as from having one or more symptoms associated with the medical condition and/or having a personal or family history associated with the medical condition. The individual may be at risk for having a medical condition, such as having a family history with the medical condition or having one or more known risk factors for the medical condition, such as high cholesterol for heart disease, being a smoker for a variety of medical conditions, having high blood pressure for heart disease or stroke, having a genetic marker associated with the medical condition, and so forth. In particular embodiments, the medical condition is a neurodegenerative disease.

In specific cases, the individual is a fetus and the fetus may or may not be suspected of having a particular nucleic acid sequence or nucleic acid expression variance compared to wild type, such sequence content or expression variance associated with a medical condition. In some cases, the fetus is at risk for a particular medical condition because of family history or environmental risk (i.e., radiation) or high-age pregnancy, for example, although the fetus may be needed to be tested for routine purposes. In such cases wherein a particular sequence(s) content or expression level is desired to be known from a fetus, a sample is taken that comprises one or more fetal cells. The sample may be a biopsy from the fetus, although in particular cases the sample is amniotic fluid or maternal blood or embryos at early stage of development.

In one aspect of the disclosure, amniotic fluid from a pregnant mother is obtained and one or more fetal cells are isolated therefrom. The fetal cell isolation may occur by routine methods in the art, such as by utilizing a marker on the surface of the fetal cell to distinguish the fetal cell(s) from the maternal cell(s). Three different types of fetal cells could exist in maternal circulation: trophoblasts, leukocytes and fetal erythrocytes (nucleated red blood cells). The most promising cell for enrichment is fetal erythrocytes, which can be identified by size column selection, followed by CD71-antibody staining or epsilon-globin chain immunophenotyping and then scanning or sorting based on fluorescence intensity, in certain embodiments.

Once the fetal cell(s) is isolated, nucleic acids are extracted therefrom, such as by routine methods in the art. The nucleic acid from the fetal cell(s) is subjected to methods of the disclosure to produce amplified cDNA that covers at least part, most, or all of the RNA, such as the transcriptome of the fetal cell(s). Following amplification, one or more sequences of the amplicons may be further amplified and also may be sequenced, at least in part, or may be subjected to microarray techniques. In specific embodiments, a SNV is assayed for, and the results of the assay are utilized in determination of whether or not the corresponding fetus has a particular medical condition or is susceptible to having a particular medical condition, for example. In specific cases, the fetus may be treated for the medical condition or may be subjected to methods of prevention or delay of onset of the medical condition, and this may occur in utero and/or following birth, for example.

Although the fetal sample may be assayed for the presence of a SNV, in particular embodiments the fetal sample is assayed for a genetic mutation associated with any particular medical condition. Examples of genes associated with prenatal medical conditions that may be assayed for include one or more of the following: ACAD8, ACADSB, ACSF3, C7orf10, IFITM5, MTR, CYP11B1, CYP17A1, GNMT, HPD, TAT, AHCY, AGA, PLOD2, ATP5A1, C12orf65, MARS2, MRPL40, MTFMT, SERPINF1, FARS2, ALPL, TYROBP, GFM1, ACAT1, TFB1M, MRRF, MRPS2, MRPS22, MRPL44, MRPS18A, NARS2, HARS2, SARS2, AARS2, KARS, PLOD3, FBN1, FKBP10, RPGRIP1, RPGR, DFNB31, GPR98, PCDH15, USH1C, CERKL, CDHR1, LCA5, PROM1, TTC8, MFRP, ABHD12 CEP290, C8orf37, LEMD3, AIPL1, GUCY2D, CTSK, RP2, IMPG2, PDE6B, RBP3, PRCD, RLBP1, RGR, SAG, FLVCR1, ZNF513, MAK, NDUFB6, TMLHE, ALDOA, PGM1, ENO3, LARS2, ATP7A, ATP7B, TNFRSF11B, LMBRD1, MTRR, FAM123B, FAM20C, ANKH, TGFB1, SOST, TNFRSF11A, CA2, OSTM1, CLCN7, PPIB, TCIRG1, SLC39A13. COL1A2, TNFSF11, SLC34A1, NDUFAF5, FOXRED1, NDUFA2, NDUFA8, NDUFA10, NDUFA11, NDUFA13, NDUFAF3, SP7, NDUFS1, NDUFV3, NUBPL, TTC19, UQCRB, UQCRQ, COX411, COX412, COX7A1, TACO1, COL3A1, SLC9A3R1, CA4, FSCN2, BCKDHA, GUCA1B, KLHL7, IMPDH1, PRPF6, PRPF31, PRPF8, PRPF3, ROM1, SNRNP200, RP9, APRT, RD3, LRAT, TULP1, CRB1, SPATA7, USH1G, ACACB, BCKDHB, ACACA, TOPORS, PRKCG, NRL, NR2E3, RP1, RHO, BEST1, SEMA4A, RPE65, PRPH2, CNGB1, CNGA1, CRX, RDH12, C2orf71, DHDDS, EYS, IDH3B, MERTK, PDE6A, FAM161A, PDE6G, TYMP (ECGF1), POLG (POLG1, POLGA), TK2, DGUOK (dGK), SURF1, SCO2 (SCO1L), SCO1, COX10, BCS1L, ACADM, HADHA, ALDOB, G6PC (GSD1a), PAH (PH), OTC, GAMT, SLC6A8, SLC25A13, CPT2, PDHA1, SLC25A4 (ANT1), C10orf2 (TWINKLE), SDHA, SLC25A15, LRPPRC, GALT, PMM2, ATPAF2 (ATP12), GALE, LPIN1, ATP5E, B4GALT7, ATP8B1 (ATPIC, PFIC), ABCB11 (ABC16, PFIC-2, PGY4), ABCB4 (GBD1, MDR2, PFIC-3), MPV17 (SYM1), TIMM8A (DDP, MTS), CPS1, NAGS, ACADVL, SLC22A5 (OCTN2), CPT1A (CPT1-L, L-CPT1), CPT1B, SUCLA2, POLG2 (HP55, MTPOLB), ACADL, SUCLG1, MCEE, GAA, PDSS1 (COQ1, TPT), PDSS2 (bA5919,3), COQ2 (CL640, FLJ26072), RRM2B (p53R2), ARG1, SLC25A20 (CACT), MMACHC (cblC), FAH, MPI, GATM, OPA1, TFAM, TOMM20 (MAS20P, TOM20), NDUFAF4 (HRPAP20, C6orf66), NDUFA1 (CI-MWFE, MWFE), SLC25A3 (PHC), BTD, OPA3 (FLJ22187, MGA3), GYS2, NDUFAF2 (B17,2L, MMTN), HLCS (HCS), COX15, FASTKD2, NDUFS4, NDUFS6, NDUFS3, MMAA (cblA), MUT, NDUFV1, MOCS1, NDUFS7 (PSST), TAZ (BTHS, G4,5, XAP-2), MOCS2, COX6B1 (COXG), HADHB, MCCC1 (MCCA), MCCC2 (MCCB), TSFM (EF-TS, EF-Tsmt), PUS1, ISCU, AGL, SDHAF1, IVD, GCDH, ADSL, DARS2, RARS2, TMEM70, ETHE1, PC, JAG1, MRPS16, PCCA, PCCB, COQ9, LDHA, PYGL, GALK1, PYGM, PGAM2, TUFM, TRMU, PFKM, GBE1, SLC37A4, GYS1, ETFDH, NDUFS8, CABC1 (ADCK3), ETFA, ETFB, DBT, SLC25A19, MMADHC, PDP1, PDHB, ACAD9, AUH, DLAT, PDHX, ACADS, NDUFS2, FBP1, NDUFAF1 (CIA30, CGI65), YARS2, SUCLG2, TCN2, CBS, PHKB, PHKG2, PHKA1, PHKA2, LIPA, ASL, HPRT1, OCRL, PNP, TSHR, ADA, ARSB, ALDH5A1, PNP, AMT, DECR1, HSD17B10, IYD, IL2RG, MGME1, HMGCL, IQCB1, OTX2, KCNJ13, CABP4, NMNAT1, ALG2, DOLK, ABCD4, ALDH4A1, ALG1, GPR143, UBE3A, ARX, GJB2 (CX26, NSRD1), APC, HTT, IKBKG (NEMO), DMPK, PTPN11, MECP2, MECP2, RECQL4, ATXN1, ATXN10, RMRP, CDKL5, PLP1, GLA, DMD, RUNX2, PLP1, CHD7, ASS1, AIRE, EIF2B, LDLR, HPRT1, RPS19, LMX1B, COL10A1, CRTAP, LEPRE1, PORCN, ASL, CFTR, ARSA, IDUA, IDS, MYO7A, GLANS, GALC, KRAS, SOS1, RAF1, AR, PTEN, BLM, SLC9A6, HRAS, GJC2 (GJA12), NPC1, NPC2, FMR1, FMR1, PLOD1, COL2A1, COL5A1, COL5A2, ABCA4, FOXG1, TINF2, USH2A, CDH23, CLRN1, CREBBP, ABCA4, POU3F4, NRAS, CHRNA7, FOXF1, MEF2C, DHCR7, RAI1, VHL, TYR (OCAIA), OCA2 (BEY, BEY1, BEY2, EYCL), TYRP1 (b-PROTEIN, CATB, GP75, SLC45A2 (AIM-1), PCDH19, SHOC2, BRAF, MAP2K1, MAP2K2, HEXA, STXBP1, ALDH7A1, SLC2A1, WDR62, MAGEL2, SDHB, and FH.

### IV. Sample Processing and Nucleic Acids from Subcellular Material of the Disclosure

One or more samples comprising subcellular material from an individual being tested with methods of the disclosure may be obtained by any appropriate means. The sample may be processed prior to steps for extracting the nucleic acid, in certain embodiments. The sample may be fresh at the time the nucleic acid is extracted, or the sample may have been subjected to fixation or other processing techniques at the time the nucleic acid is extracted.

The sample may be of any kind. In embodiments wherein cellular material of interest is comprised in the sample, the subcellular material may be isolated based on a unique feature of the desired cell or cells or subcellular structures, such as a protein expressed on the surface of the cell or associated with a subcellular structure. In embodiments wherein a fetal cell is isolated based on a cell marker, the cell marker may be CD71 or epsilon-globin chain, etc. In embodiments wherein a cancer cell is isolated based on a cancer marker, the cell marker may be ER/PR, EGFR, KRAS, BRAF, PDFGR, UGT1A1, EphA2, HER2, GD2, Glypican-3, 5T4, 8H9, αᵥβ6 integrin, B cell maturation antigen (BCMA) B7-H3, B7-H6, CAIX, CA9, CD19, CD20, CD22, kappa light chain, CD30, CD33, CD38, CD44, CD44v6, CD44v7/8, CD70, CD123, CD138, CD171, CS1, CEA, CSPG4, EGFR, EGFRvIII, EGP2, EGP40, EPCAM, ERBB3, ERBB4, ErbB3/4, FAP, FAR, FBP, fetal AchR, Folate Receptor a, GD3, HLA-AI, HLA-A2, IL11Ra, IL13Ra2, KDR, Lambda, Lewis-Y, MCSP, Mesothelin, Muc1, Muc16, NCAM, NKG2D ligands, NY-ESO-1, PRAME, PSCA, PSC1, PSMA, ROR1, Sp17, SURVIVIN, TAG72, TEM1, TEM8, carcinoembryonic antigen, HMW-MAA, VEGF receptors, MAGE-A1, MAGE-A3, MAGE-A4, CT83, SSX2, XIAP, cIAP1, cIAP2, NAIP, Livin, etc.

The isolated subcellular structures can be lysed by incubating the cell in RNase-free lysis buffer with surfactant (i.e. Trion-X100, tweet-20, NP-40, etc.), a reducing agent (i.e. dithiothreitol, etc.), and an RNase inhibitor (i.e. RNaseOUT, etc.). Furthermore, cells or subcellular structures can be lysed in the presence of primers described in the disclosed method.

### V. Kits of the Disclosure

Any of the compositions described herein or similar thereto may be comprised in a kit. In a non-limiting example, one or more reagents for use in methods for amplification of nucleic acid may be comprised in a kit. Such reagents may include enzymes, buffers, nucleotides, salts, primers, and so forth. The kit components are provided in suitable container means. In some embodiments, cellular material including at least subcellular structures are of a desired type and are provided in the kit.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### General Embodiments

Synapses are crucial structures that mediate signal transmission between neurons in complex neural circuits, and they display considerable morphological and electrophysiological heterogeneity. So far there is still not a high-throughput method to profile the molecular heterogeneity among individual synapses. The present disclosure provides a droplet-based SC and SSS total-RNA-seq method that allows the transcriptome profiling of individual neurites, primarily composed of synaptosomes. The transcriptome of single synaptosomes is referred to herein as a synaptome. In the synaptome profiling of both human and mouse brain samples, different subclusters were detected among synaptosomes and the association between the subclusters of synaptosomes and the subtypes of neurons was identified. In addition, the landscape of local splicing that occurred in synapses was characterized. The synaptome profiling was further extended to synaptopathy in an Alzheimer's disease (AD) mouse model. As a result, the inventors discovered the novel AD-associated synaptic gene expression changes that cannot be detected by single-nucleus transcriptome profiling. Overall, the results show that this platform, referred to herein as Multiple-Annealing-and-Tailing-based Quantitative scRNA-seq in Droplets (MATQ-Drop), provides a high-throughput single-synaptosome transcriptome profiling tool that will facilitate future discoveries in neuroscience.

### Example 2

### The Chemistry of MATQ-Drop

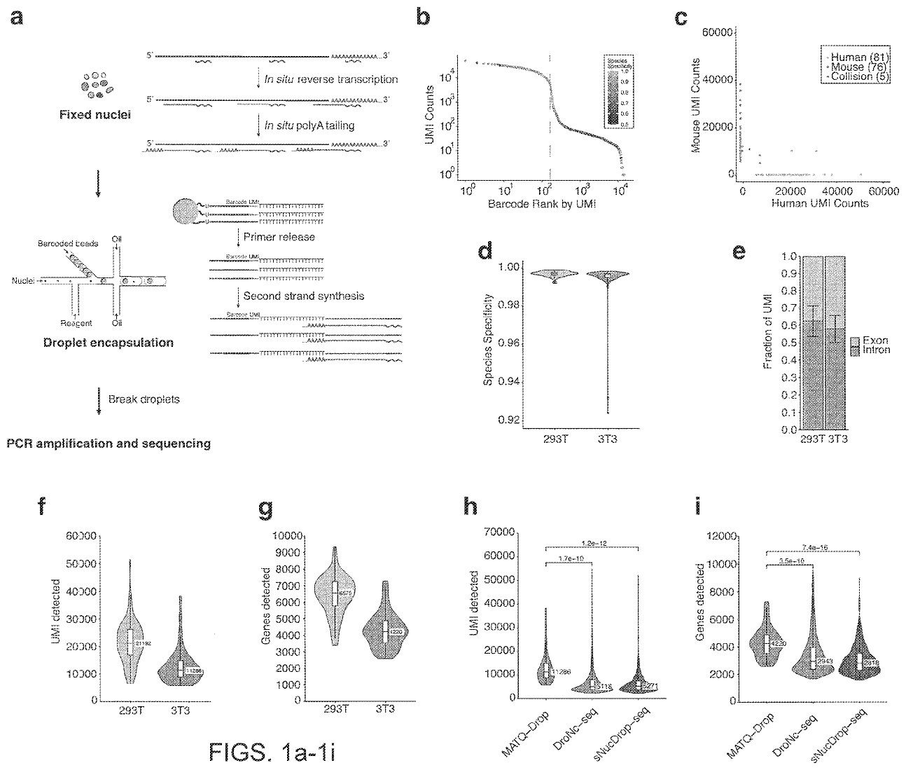

In the chemistry of MATQ-Drop (FIG. 1a), 3% paraformaldehyde (PFA) was first applied to fix the nuclei. After the crosslinking, the nucleus membrane was permeabilized and ten cycles were performed of multiple annealing with MALBAC primers^{6, 12}, which allow efficient hybridization to the internal regions of the transcripts (FIG. 1a). As a result, besides the reverse transcriptions initiated from the poly-adenylated tails at the 3′ end of the transcripts, significant portions of reverse transcriptions were also initiated at the internal regions of transcripts, which warrants the efficient total RNA capture. After the reverse transcription step, the excessive MALBAC primers were washed away. Then, in situ poly adenine tailing was performed for the cDNA molecules, which is referred to as the dA-tailing step. Next, the processed nuclei were washed and microfluidic platforms were used to encapsulate single nuclei together with the barcoded dT20 hydrogel beads in droplets for multiplexed second strand synthesis. The barcoded dT20 hydrogel beads were prepared following the procedure described in the inDrop platform¹⁰.

It is worth noting that different from the UV-triggered release of the barcoded oligos from the beads in the inDrop platform, here the inventors introduced enzymatic release chemistry. In this chemistry, a deoxyUridine base was introduced in the sequence near the 5′ end of the barcoded oligos. In the droplet reaction buffer for the second-strand synthesis, the USER enzyme was included that can cut the oligos at the deoxyUridine site. As a result, upon droplet encapsulation, the dT20 oligos with cell barcodes were efficiently released from the beads. Next, RNA digestion was performed followed by heat decrosslinking to release cDNA from the nuclei. The barcoded dT20 primers then hybridized to the poly A tail of the cDNA molecules to initiate the second-strand synthesis. After the second strand synthesis was completed, the droplets were broken and the aqueous phase was collected, followed by the PCR reaction to amplify the library for next-generation sequencing.

To validate the successful single-cell barcoding in MATQ-Drop, the inventors have performed the standard species-mixing experiment as a control. Equal numbers of fresh human HEK293T and mouse NIH/3T3 cells were mixed and then lysed into nuclei. With the fixed nuclei, the MATQ-Drop assay was performed as described above. Here a small aliquot of droplets was used to generate the sequencing library for technical evaluation. As shown in FIG. 1b, 162 unique high-quality cell barcodes were identified. Based on the species specificity, the inventors unambiguously assigned them to 81 human 293T nuclei, 76 mouse 3T3 nuclei, and 5 collision events (FIG. 1c). For each assigned cell barcode, there was high species specificity of UMI, as shown in FIG. 1d (99.7% for 293T nuclei and 99.4% for 3T3 nuclei). In addition, the 162 cell barcodes covered 89% of all uniquely mapped reads, confirming an extremely low cross-barcode contamination rate. For the single-cell Total-RNA-seq data generated by MATQ-Drop, there was no significant UMI inflation.

The major technical advantage of MATQ-Drop, in comparison to matured mRNA-based platforms such as 10× Genomics Chromium, is that one can effectively detect nascent RNAs using the reads mapped to intronic regions (FIG. 1e). Regarding gene detection sensitivity, at the average sequencing depth of ˜70,000 raw reads per single nucleus, there was a median of 21,192 UMIs and 6,575 genes for single 293T nuclei, and 11,286 UMIs and 4,220 genes for single 3T3 nuclei (FIGS. 1f-g). As shown in FIGS. 1h-l, the gene detection of MATQ-Drop is significantly higher than the sensitivity of other single-nucleus RNA-seq methods^{8, 13}. To further extend the benchmark comparison between MATQ-Drop and 10× Genomics Chromium for cell atlas construction with tissue samples, an equal footing comparison was also performed using mouse hippocampus samples described below.

### Synaptome Profiling of the Human Hippocampus and Prefrontal Cortex Detects the Subtypes of Synapses

So far, the major approach in transcriptome profiling of synapses is based on bulk samples¹⁴. Noticeably, micro-dissected neurites were used to profile the transcriptome of synapses localized at specific regions of rat hippocampus samples¹⁵. Here, with the development of MATQ-Drop, one can profile the transcriptome of individual synaptosomes in contrast to the bulk approach.

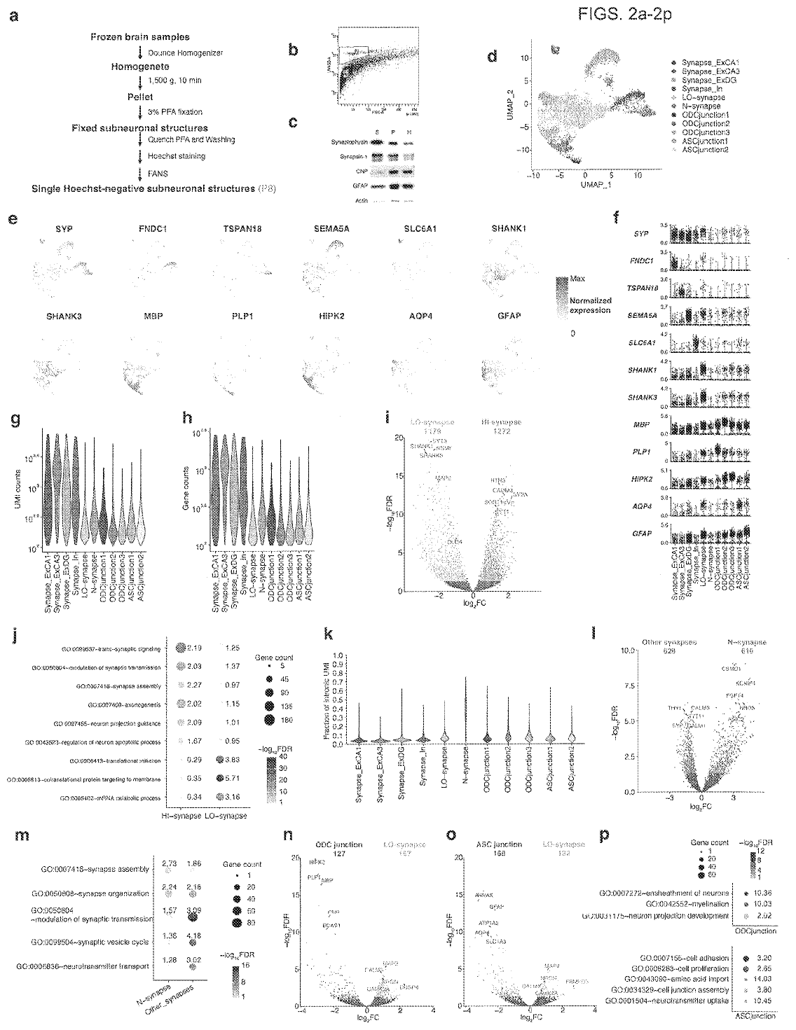

The test utilized frozen human brain samples. To isolate synaptosomes from human brain samples, the inventors first ground out the frozen brain tissue using a Dounce homogenizer. FACS was then performed to enrich Hoechst-negative subcellular structures with sizes smaller than 5 μm (FIG. 2a-b). To validate the synaptosome isolation procedure, the inventors confirmed the enrichment of synaptic proteins synaptophysin and synapsin-1 in the Hoechst-negative subcellular structures using Western Blot (FIG. 2c). In addition, immunostaining was performed for the Hoechst-negative particles using presynapse marker Synaptophysin and postsynapse marker PSD95. Using flow cytometry analysis, 60.1% of Hoechst-negative particles were Synaptophysin-positive, and 38.1% were PSD95-positive. Next, double-positive particles (34.6%) were sorted out and transcriptome profiling was performed. When its transcriptome data was combined with the transcriptome data of the total Hoechst-negative particles of the same sample, there was a complete overlap between Hoechst-negative particles and double-positive particles, indicating that the vast majority of Hoechst-negative particles are synaptosomes and neuron-glia junctions. Neuron-glia junctions were reported to express synapse proteins¹⁶, therefore, they were enriched in the Synaptophysin and PSD95 double-positive population.

On the other hand, the inventors also sorted out the double-negative particles (36.4%) and performed transcriptome profiling. As a result, the corresponding transcriptome had extremely low RNA abundance per particle, equivalent to 4% of RNA yield compared to the double-positive population. Hence, when the transcriptome was profiled of all Hoechst-negative particles, the double-negative particles are effectively filtered out by RNA abundance cutoff and do not contribute to the synaptome. Therefore, the unbiased profiling of the Hoechst-negative population authentically represented the transcriptome of synaptosomes and neuron-glia junctions.

In specific embodiments, the main reason for conducting this rapid isolation of synaptosomes is to preserve RNA quantity and quality. In comparison to the rapid isolation procedure, synaptome profiling using synaptosomes isolated from the standard gradient centrifugation-based enrichment method (Described in the mouse hippocampus data below) was performed. As a result, a significant reduction of gene detection was observed, leading to the poor resolution of synaptosome clustering.

For two human hippocampus samples, the inventors generated the transcriptome of 10,428 single subcellular structures (FIG. 2d), and we observed 11 major clusters corresponding to different types of neurite structures. The batch effects between samples were undetectable. The inventors specifically requested the dentate gyrus regions of the hippocampus samples. In FIG. 2e-f, they annotated these clusters as subtypes of synapses and neuron-glia junctions based on the well-known molecular markers enriched in those subcellular structures. In total, 6 synapse-associated clusters were assigned: four synapse clusters with high RNA abundancy (denoted as HI-synapses), one synapse cluster with lower RNA abundancy (denoted as LO-synapses) (FIGS. 2g-h), and another synapse cluster containing relatively higher nascent transcripts (denoted as N-synapses) (FIG. 2k).

When the synaptome profile was compared with single-nucleus transcriptome profiles described below, the four HI-synapse clusters could be associated with excitatory neurons in CA1, CA3, and DG regions and inhibitory neurons, respectively. The inhibitory HI-synapse cluster (Synapse_In in FIG. 2d) can be further classified into three subtypes by additional subclustering analysis. When the synaptome of two human prefrontal cortex (PFC) samples was profiled, similar clusters of HI-synapses, LO-synapses, and N-synapses were observed. The HI-synapses can also be clustered into excitatory and inhibitory subtypes.

### Differential Gene Expression Between Different Subtypes of Synapse Clusters

Next, a differentially expressed gene (DEG) analysis was performed to identify transcriptomic differences between the HI-synapses and the LO-synapses for the hippocampus synaptome (FIG. 2i) and the PFC synaptome. The inventors identified 1,272 and 807 HI-synapse-enriched genes (abs(log₂FC)>log₂1.3, FDR<0.05) in the hippocampus and PFC, respectively, both including well-established synaptic vesicle genes (SYT1, SYP, SV2A, and SORT1) 17. Next, 1,179 and 855 LO-synapse-enriched genes were identified in the hippocampus and PFC, respectively. Among the enriched genes in the LO-synapses, the dendrite marker gene MAP218, the well-known postsynaptic scaffold genes SHANK1, SHANK3, and DLG419, and the postsynaptic gene SYT320 were noticed. The differential marker genes show the enrichment of presynaptic transcriptomic features in the HI-synapses and the enrichment of postsynaptic transcriptomic features in the LO-synapses. In the enriched functionals and pathways of the HI-synapse cluster, there was synaptic signaling and axonogenesis in both hippocampus (FIG. 2j) and PFC. For the LO-synapse cluster, the protein synthesis and mRNA catabolism-related pathways are enriched (FIG. 2j), suggesting high protein synthesis activities and turnover rates exist in the postsynapses.

Interestingly, while the vast majority of synapses displayed a low intron fraction (7.85% on average), one cluster (N-synapses) exhibited a significantly higher intron fraction (30.79% on average, FIG. 2k). Through the DEG analysis between N-synapses and the rest of the synapses (FIG. 2l), the genes enriched in N-synapse were overrepresented in synapse assembly and synapse organization gene sets (FIG. 2m). In contrast, the genes involved in synaptic signaling were overrepresented in other clusters of synapses (FIG. 2m). The results indicate that N-synapses represent the immature synapses that are in process of assembly and maturation. The significantly higher percentage of intronic reads in the N-synapses also buttresses the important roles unspliced nascent RNA and the related local splicing in the synaptic assembly and maturation process.

### Synaptome Profiling of Human Hippocampus and Prefrontal Cortex Detects Neuron-Glia Junctions

Besides the clusters of synaptosomes, two major cell-cell junctions formed between neurons and glial cells: neuron-oligodendrocyte junctions (ODC junction), and neuron-astrocyte junctions (ASC junction) in the hippocampus (FIG. 2d) and PFC. Both non-compact myelin gene CNP and compact myelin genes, PLP1 and MBP, were highly expressed in ODC junctions (FIG. 2n). It is worth noting that the upregulated genes in the ODC junctions are enriched in the myelination process (FIG. 2p), which is consistent with the well-known axon-ODC signaling related to the myelination process^{21, 22}. More importantly, the detection of transcripts in ODC junctions in the data suggests the importance of local translation at the ODC junctions during myelination. This indication of local translation is also consistent with the recent study by Wake et al.²³.

In the ASC junctions, there was local enrichment of ASC-specific genes, for example, GFAP, ATP1A2, AQP4, and SLC1A3 (FIG. 20). These upregulated genes are enriched in cell adhesion, proliferation, and neurotransmitter uptake pathways (FIG. 2p). Consistent with the observation of transcripts enriched in the ASC junctions, local translation has also been recently observed in astrocyte peripheral processes²⁴. Overall, the transcriptome profiling of neuron-glia junctions allows the comprehensive identification of locally translated genes in the cell-cell junctions between neurons and glial cells. The functional roles of these genes are worth future investigation.

### Effective Construction of Cell Atlas for Human Hippocampi and Prefrontal Cortex Using Nascent RNAs

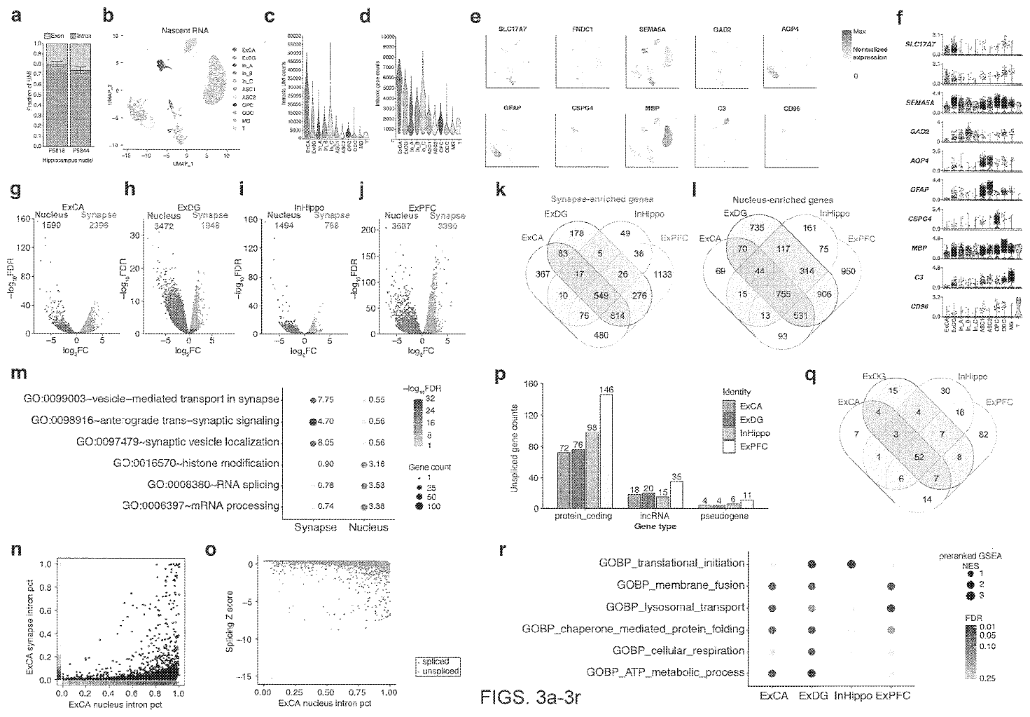

To identify the connection between different subtypes of synaptosomes and different subtypes of neurons, next, the inventors applied MATQ-Drop to profile the total-RNA based transcriptome for 8,112 single nuclei isolated from two dissected frozen human hippocampi. First, in the single-nucleus transcriptome data, the portion of reads that represented nascent RNAs in the brain samples was significantly higher than that in the cell line samples (FIG. 3a). The inventors observed that 78% of the UMIs were mapped to intronic regions in the brain samples (FIG. 3a) in contrast to 63% of intronic reads in the cell lines (FIG. 1e). Next, the gene expression matrix was calculated based only on the unspliced transcript sequence with the reads mapped to the intron regions, which is different from the commonly used spliced transcript with reads mapped to the exon regions.

Using the nascent RNA-based gene expression matrix, its performance was evaluated in constructing a cell atlas for human hippocampus samples the inventors profiled. Here the standard Seurat k-nearest neighbor graph-based unsupervised clustering was used²⁵. In FIG. 3b there was identification of the following 10 primary clusters in the hippocampal nuclei: 2 excitatory neuronal subtypes from the Cornu Ammonis region (ExCA) and dentate gyrus (ExDG), respectively; 3 inhibitory neuronal subtypes (In_A, In_B, In_C); 4 glial cell types, including two subtypes of astrocytes (ASC1-2), oligodendrocyte precursor cells (OPC), oligodendrocytes (ODC), and microglia (MG). No batch-to-batch variations were observed. In terms of detection sensitivity, the UMI and gene detection are shown in FIG. 3c-d. The markers of each cluster were also consistent with well-established cell type-specific markers (FIGS. 3e-f), suggesting the robust cell-typing using a nascent-transcript based gene expression matrix.

Similar to the hippocampus, the cell atlas was constructed for the human PFC sample of the same individuals. With the profiling of 939 single nuclei, there was identification of 15 primary clusters with high confidence, which included 6 excitatory neuronal subtypes (Ex1-6), 4 inhibitory neuronal subtypes (In1-4), 4 glial cell types (including astrocytes (ASC), oligodendrocyte precursor cells (OPC), oligodendrocytes (ODC) and microglia (MG)), and endothelial cells (END). The markers of each cluster were also consistent with the standard cell-type-specific markers. Based on the expression of the previously reported layer-specific markers²⁶, the six excitatory neuron subtypes were assigned to different cortical layers. Among the inhibitory neurons from both regions, we identified 8 subtypes with additional sub-clustering analysis. The unique combinations of marker genes were detected in the subtypes of inhibitory neurons.

### Differentially Expressed Gene Analysis Between Synapses and the Associated Nuclei in Human Samples

Now with the single nucleus transcriptome data from the same tissues of the synaptome, the inventors were able to connect the subclusters in the synaptome to different neuronal nucleus types based on the shared marker genes (FIG. 2d). Three HI-synapse clusters could be connected to the excitatory neurons in hippocampal CA1, CA3, and DG regions, and another HI-synapse cluster could be connected to inhibitory neurons (FIG. 2d). Next, the differential patterns of RNA expression was investigated between the synaptic clusters and the associated nuclei. Considering the dominance of matured mRNA in the synapses, here the inventors used the exon-based gene expression matrix for DEG analysis between synapses and nuclei.

In total, an average of 2,126 synapse-enriched genes and 2,548 nucleus-enriched genes were identified (FIGS. 3g-j). In FIGS. 3k-l, the overlapped genes between different neuronal subtypes was examined. There were a total of 4,099 synapse-enriched genes and a total of 4,848 nucleus-enriched genes. 549 synapse-enriched genes and 755 nucleus-enriched genes were shared by all four neuronal subtypes, respectively (FIGS. 3k-l). Next, in the functional analysis, the 549 shared synapse-enriched genes were overrepresented in the pathways directly related to synaptic signaling (FIG. 3m). In contrast, the 755 nucleus-enriched genes were overrepresented in the epigenetic regulation and RNA processing pathways (FIG. 3m).

### Local Splicing Landscape in Different Subtypes of Synaptosomes

Studies have shown that the genes with retained introns are crucial for the intraneuronal transport of the transcript²⁷. Furthermore, synaptic alternative splicing is also vital for quick modulation of synaptic functions²⁸⁻³¹. Next, based on nascent RNA detection in MATQ-Drop data, intron retention was characterized for different clusters of synapses. As a result, one can determine whether the synaptic splicing pattern is the same across different synapse clusters. For all four clusters in HI-synapses, a long tail of outliers with clear evidence of intron retention was observed (FIG. 3n). It is worth noting that 84.5% of synaptic transcripts were already spliced (intron percentage <5%). Next, the intron percentage was compared between synapses and nuclei for each gene and ranked based on their splicing Z score (FIG. 3o). The inventors then performed the GSEA with the genes preranked by the splicing Z score. The essential cellular functions such as translation, protein folding, and metabolism were significantly enriched in the fully spliced genes (FIG. 3r). This result confirms that the fully spliced genes detected in synapses are mainly responsible for basic cellular functions. Based on the splicing Z score, the unspliced genes with statistical significance were identified, and in total, the 256 genes from different HI-synapse subtypes were detected, including 49 lncRNA, 11 pseudogenes, and 196 protein-coding genes (FIG. 3p). Interestingly, only 41 unspliced protein-coding genes were shared by all 4 synapse subclusters (FIG. 3q). This result indicates that a significant portion of local synaptic splicing is uniquely associated with specific synapse types.

### Synaptome Profiling of Freshly Prepared Mouse Hippocampus

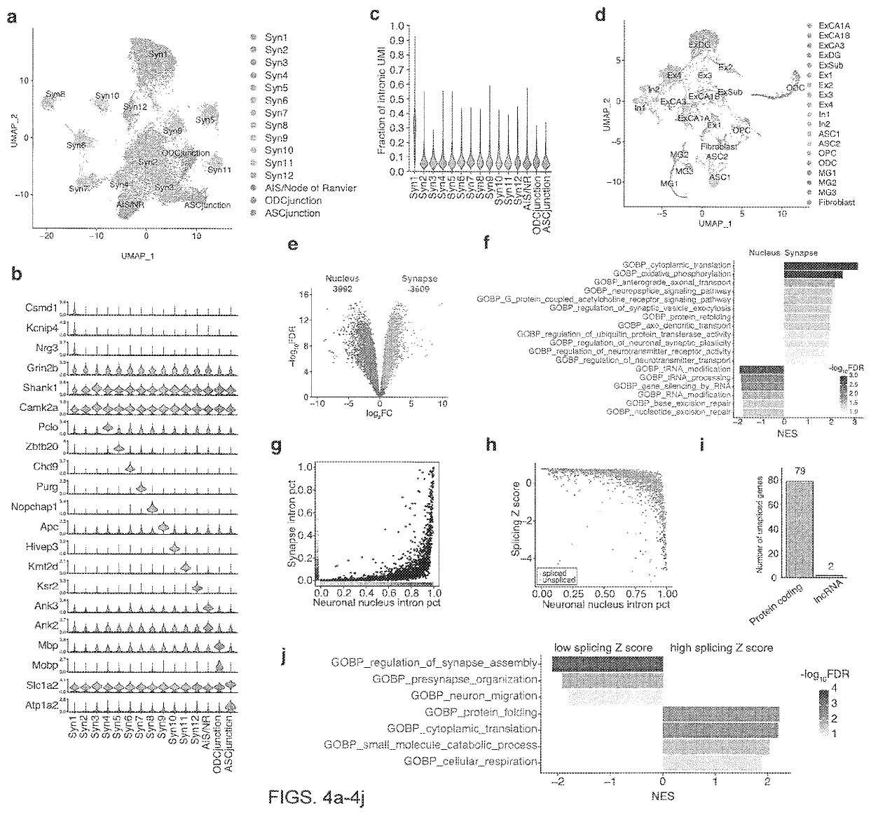

The human brain samples often had long postmortem intervals (12 and 13 hours, respectively, for the two brain samples we sequenced), which could lead to the decay of transcripts and distort the synapse clusters. To avoid this potential sample bias, next the inventors applied MATQ-Drop to profile the synaptome and single-nucleus transcriptome for the freshly prepared mouse brain samples. The synaptome of the mouse hippocampus was profiled and the analysis performed using the same unsupervised clustering pipeline used for the human samples. Interestingly, 15 primary clusters were identified, among which 12 clusters were synapse-associated (FIGS. 4a-b). In contrast, only 6 synapse-associated clusters were observed in human hippocampus samples. The potential reason for this observation is that mouse brain samples were freshly prepared right after the sacrifice of the mice. Therefore, synapse states were likely better preserved in the mouse sample.

Among the 12 synapse-associated clusters, the Syn1 cluster exhibits a 3.5-increase of nascent RNA proportion compared to the rest of synapses (average intronic fraction 29.9% versus 8.5%, FIG. 4c), hence this cluster is likely the mouse counterpart of human N-Synapse. In Syn2 and Syn4 clusters, the inventors observed the upregulation of Grin2b, Pclo, and Bsn (Pclo and Bsn are known pre-synaptic scaffold genes), similar to the enriched genes observed in human HI-Synapse. In contrast, in the Syn3 cluster, there was upregulation of postsynaptic genes, including Shank1 and Shank3, similar to the enriched genes observed in human LO-Synapse (FIGS. 4c and 2d).

Besides the overrepresentation of presynaptic features in Syn2 and Syn4 clusters and postsynaptic features in Syn3 cluster, there was observed additional synapse subclusters that are defined by specific markers: Syn5: Zbtb20, Syn6: Chd9, Syn7: Purg, Syn8: Nopchap1, Syn9: Apc, Syn10: Hivep3, Syn11: Kmt2d, Syn12: Ksr2 (FIGS. 4a-b). Among their marker genes, mutations in Zbtb20 have been shown to affect the synaptic structures by altering ZBTB20 protein localization in subneuronal compartments³²; Purg (detected in Syn7) was reported to display strong and early upregulation during synaptogenesis in primary mouse hippocampal neurons³³; Ksr2 (detected in Syn12) contributes to calcium-mediated ERK signaling³⁴. In addition to synapse-associated clusters, there was observed axon initial segments and nodes of Ranvier (AIS/NR cluster), and neuron-glia junctions including ODC junctions and ASC junctions (FIGS. 4a-b).

In contrast to the clear difference in RNA abundance between HI-Synapses versus LO-Synapses detected in the human brain, we did not observe such a discrepancy in mouse brains. In one embodiment, it is caused by species differences between human and mouse, or by different RNA decay rates between presynapses and postsynapses. If the postsynapses have a much higher RNA decay rate than the presynapses, then with the long post-mortem intervals for the human samples used, a significant portion of RNA in postsynapse might have been decayed before they can be captured by MATQ-Drop.

### Mouse Hippocampus Synaptome Using Synaptosomes Enriched by Density Gradient-Based Approach

Next, the inventors used the freshly prepared mouse samples as the control to compare the effects of different synaptosome isolation procedures on synaptome profiling. Synaptome profiling was performed using the synaptosomes isolated from the standard sucrose density gradient-based ultra-centrifugation protocol. In comparison to the direct sorting-based procedure, the inventors observed 53% fewer genes detected per synaptosome (median 146 genes versus 306 genes), which is likely due to RNA decay and the leakage during the extensive processing time without PFA fixation. While there was some evidence of the regional distribution for a few clusters including Syn1 (Kcnip4), Syn6 (Chd9), and Syn8 (Nopchap1), the overall clustering results has low-resolution with certain ambiguity.

### Connect the Synapse Clusters to Neuron Subtypes

To compare the synaptome to the nucleus transcriptome, next the inventors performed single nucleus transcriptome profiling for the same mouse hippocampus. Based on the nascent RNA expression matrix, there was identification of 9 subtypes of excitatory neurons from different subregions, 2 subtypes of inhibitory neurons, astrocytes, oligodendrocyte progenitor cells, oligodendrocytes, microglia, and fibroblasts (FIG. 4d) and an average of 83% of UMIs detected can be attributed to introns.

Similar to what was done for human samples, next the inventors compared the single nucleus transcriptome to the synaptome to identify the connection between synapse clusters and neuron subtypes. Interestingly, there was not identification of statistically significant connections. This result supports that the inventors captured different synaptic states with the synaptosomes prepared from fresh mouse brain samples. One can investigate among different synaptic states (synapse clusters) whether one can identify the subclusters associated with neuron subtypes. To do so, one could use the highly variable genes across neuronal nuclei as the coordinates for supervised clustering analysis. There was observed the evident association between the distribution of synaptosomes and different neuronal subtypes. However, the subclusters are less separated, likely because they share the features of the same synaptic states. Based on the mouse synaptome data, there are two layers of synapse heterogeneities: the first layer is associated with synaptic states and the second is associated with neuron subtypes.

### Differentially Expressed Gene Analysis Between Synapses and the Associated Nuclei and Local Splicing Landscape in Mouse Hippocampus

Next, the inventors performed the DEG analysis between synapses and nuclei, and 3,609 synapse-enriched genes and 3,992 nucleus-enriched genes were identified (FIG. 4e). Consistent with the observation in human samples, synapse-enriched genes were overrepresented with synaptic signaling and protein synthesis pathways. In contrast, the nucleus-enriched genes were overrepresented with gene regulation, RNA processing, and DNA repair pathways (FIG. 4f).

For the synaptic transcript splicing pattern, the same intron retention analysis was performed as the human synaptome, and only a small percentage of unspliced synaptic transcripts were observed (81 out of 2015, 4%), including 79 protein-coding genes and 2 lncRNAs (FIGS. 4g-i). When the inventors performed the GSEA for the genes preranked by splicing Z score, on one end of the enrichment, the spliced transcripts were enriched for basic cellular activities such as protein synthesis and metabolism; on the other end of the enrichment, the unspliced transcripts were enriched for synapse assembly, organization, and neuron migration pathways, suggesting the important role of local splicing in synaptogenesis (FIG. 4j).

### Characterization of Synaptopathy in Alzheimer's Disease (AD) by Synaptome Profiling

As a hallmark of AD, β-amyloid plaques are also known for impairing synaptic function and inducing synaptopathy. It has been shown that β-amyloid plaques can induce an inflammatory response that activates microglia to prune synapses^{35, 36} and block post-synaptic NMDA receptors and, therefore, suppress trans-synaptic signaling³⁷. Current profiling of transcriptomic changes associated with AD has only been done with single-nucleus RNA-seq^{38, 39}. Here to characterize the synaptome changes in AD and examine whether different synapse subtypes have different responses to β-amyloid plaques, the inventors profiled the transcriptome of 6,989 single nuclei and 20,456 single Hoechst-negative particles isolated from two wildtype and two 5×FAD mice.

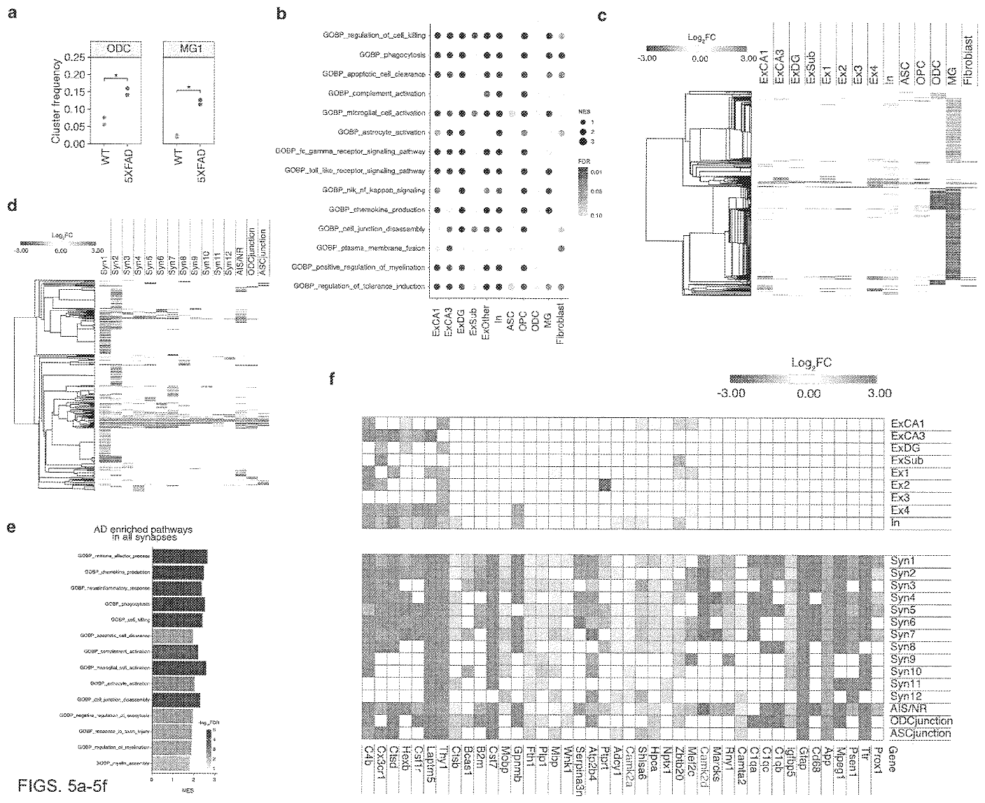

From single-nucleus transcriptome data, there was a 2.3-fold overrepresentation of oligodendrocytes compared to WT in terms of cell-type composition. This result is likely due to the response to axon demyelination (FIG. 5a). There was a 5.4-fold increase of the major microglia subtype (MG1) in the 5×FAD mice, indicating an activated inflammatory response (FIG. 5a). These changes are consistent with the previous studies^{38, 39} Next, for each neuronal subtype and glial cell type, the inventors identified the DEGs associated with AD based on nascent RNA (FIG. 5c) and mature RNA, respectively. In particular, microglia consistently displayed the highest numbers of DEGs, suggesting more sensitive roles of these cells in disease response compared to other cell types (FIG. 5c), which are also consistent with the previous study⁴⁰. When the inventors applied the GSEA, myelination and multiple inflammatory response pathways including cell killing, complement activation, and chemokine production, were enriched in AD across various cell types (FIG. 5b). It is worth emphasizing that while similar pathways were enriched in GSEA for different cell types (FIG. 5b), the DEGs are not identical for different cell types, suggesting different response mechanisms to the amyloid pathology exist among different cell types.

Next, DEGs were identified of each cluster of synapses and neuron-glia junctions between the 5×FAD and wildtype mouse in the hippocampal synaptome (FIG. 5d). In total, 410 genes with significant DEGs (abs(log₂FC)>log₂1.3, FDR<0.05) were identified among different clusters, among which 42 genes were shared by more than half of synapse clusters and 246 genes were unique to single clusters (FIG. 5d). In line with the single-nucleus results, neuroinflammatory response, complement activation, and myelination pathways were significantly enriched in the AD synaptosomes (FIG. 5e), indicating the general inflammatory stress associated with β-amyloid plaques. In addition, the inventors also observed the enrichment of cell junction disassembly and negative regulation of exocytosis pathways, indicating synapse loss and decreased synaptic function.

For the 42 AD DEGs shared by all synapse subtypes, the corresponding gene expression changes in nuclei were plotted in FIG. 5f (nascent RNA based DEGs, Top: nuclei, Bottom: synapses). It is worth noting that 24 synapse AD DEGs cannot be detected from the nucleus transcriptome data. Furthermore, 8 genes exhibited opposite dysregulation directions from the DEG change based on the nucleus transcriptome data. Interestingly, three complement components, C1qa, C1qb, and C1qc, were all significantly upregulated in the synapses but not significant in the nuclei, indicating a potential role of local translation of these components in complement-mediated synapse pruning. It is desired to unveil how these complement component transcripts are transported to the abnormal synapses that require pruning. There was bifurcated expression of two calcium/calmodulin-dependent protein kinase II (CaMKII) genes: Camk2a and Camk2d (FIG. 5f, red labeled genes), which suggests a switch of CaMKII isoforms in AD that potentially impacted synaptic plasticity⁴¹.

Effective Construction of Cell Atlas Using Only lncRNA Species

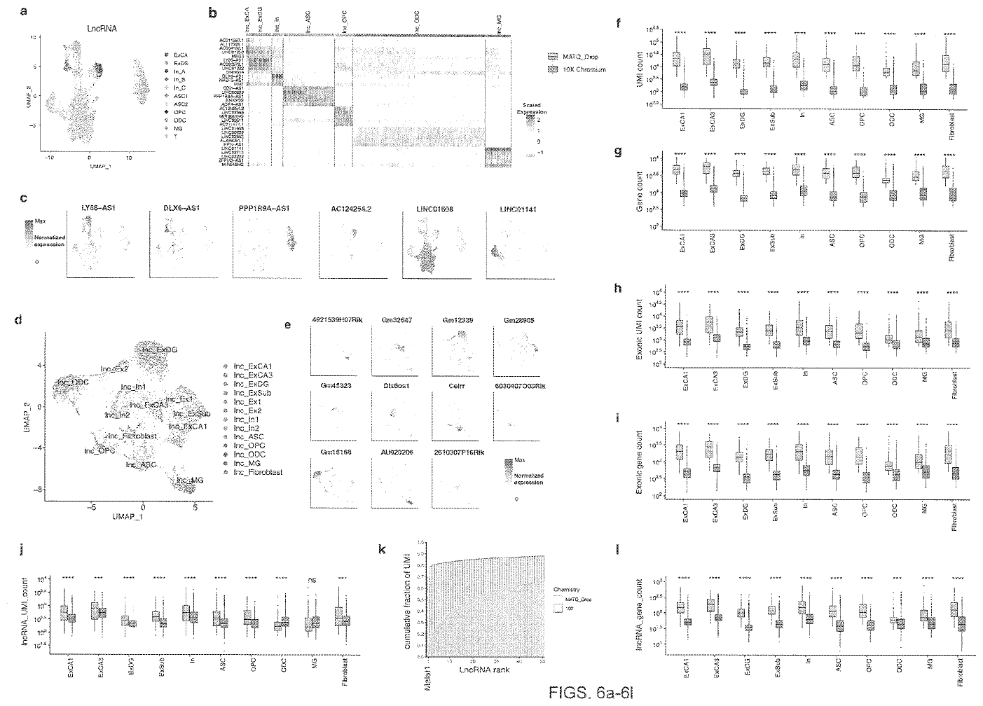

Different from mature RNA-based droplet platforms, the total RNA-based chemistry of MATQ-Drop allows the efficient detection of long non-coding RNAs (lncRNA). Next, it was examined whether one could successfully identify the cell types using only the lncRNA expression matrix. The successful construction of a cell atlas using only lncRNA species will indicate that cell-type-specific lncRNA species or cell-type-specific composition of lncRNA species exist. As shown in FIG. 6a, there was robust construction of the cell atlas for the human hippocampus by unsupervised clustering. The clustering result is also consistent with nascent transcript-based clustering. The lncRNA-based cell atlas of human PFC was also successfully constructed. Cell type-specific lncRNA markers can be systematically identified by MATQ-Drop (FIGS. 6b-c).

For the mouse hippocampus, the cell atlas was constructed using only lncRNA species (FIG. 6d). The clustering result is highly consistent between lncRNA-based and nascent RNA-based clustering (FIG. 3b). As a result, cell type-specific lncRNA markers were systematically identified (FIG. 6e). It is worth noting that lncRNAs with polyadenylated tails can also be detected using SMARTer chemistry on the Fluidigm platform⁴². However, MATQ-Drop chemistry allows the detection of the complete spectrum of lncRNAs, including those with polyadenylated tails and those without polyadenylated tails. Furthermore, the droplet platform offers higher throughput than the Fluidigm platform in identifying cell-type-specific lncRNA species.

### Benchmark Comparison Between MATQ-Drop and 10× Chromium Platform Based on Single-Nucleus Transcriptome Profiling of Mouse Hippocampus

With the MATQ-Drop based single-nucleus transcriptome data of mouse hippocampus and the recent mouse hippocampus single-nucleus transcriptome data generated on the 10× Chromium platform³⁹, next, the inventors performed an equal footing benchmark comparison between MATQ-drop and the 10× Chromium platform. When counted based on transcripts, MATQ-Drop detected a median of 16,593 UMI and 4,186 genes for single neuronal nuclei, and 9,525 UMI and 3,043 genes for glial nuclei. Both are significantly higher than the 10× Chromium data (median 1,390 UMI and 886 genes for single neuronal nuclei, or 1,142 UMI and 779 genes for single glial nuclei, FIG. 6f-g). Even when the exon-based gene expression matrix is used to compare with 10× Chromium, there is a 2.4-3.6 fold increase in UMI detection and a 2.5-3.7 fold increase in gene detection (MATQ-Drop median: neuronal nuclei 2,670 UMI and 1,640 genes, glial nuclei 1,615 UMI and 1,059 genes; 10× Chromium median: neuronal nuclei 738 UMI and 449 genes, glial nuclei 673 UMI and 424 genes, FIGS. 6h-i).

In the detection of lncRNA, the sensitivity of the 10× platform is only slightly lower than MATQ-Drop in terms of the detected UMI number (FIG. 6j). However, when the inventors examined the 10× Chromium data in details, a single lncRNA gene, Malat1, contributed to 70% of the total UMI count (FIG. 6k). This biased detection is likely due to the large portion of the AT-rich sequence in this gene, therefore, allowing more efficient internal hybridization by oligo (dT) primers than other genes. In contrast, there was not observed a significant contribution by one gene in MATQ-Drop. Overall, in terms of lncRNA gene detection, MATQ-Drop shows a 2.1-2.6 fold improvement over the 10× platform (MATQ-Drop median: neuronal nuclei 119 genes, glial nuclei 83 genes; 10× Chromium median: neuronal nuclei 46 genes, glial nuclei 40 genes, FIG. 6l). This unbiased detection of lncRNAs in MATQ-Drop is also vital for successful cell typing using only lncRNA species as described above.

### Significance of Certain Embodiments

Supported by the chemistry and sensitivity of MATQ-Drop, the transcriptome was profiled of individual synapses in high-throughput for the first time. There was successful detection of different subtypes of synaptosomes and other types of junctions between neurons and nonneuronal cells. The enrichment of different functional pathways between synaptosome subtypes was also observed, supporting the existence of phenotypical heterogeneity between different synaptosomes. Different synaptosome subtypes could be connected to different types of neurons. Besides synaptome profiling, MATQ-Drop can also be used to construct cell atlas. More importantly, it was shown that one could successfully construct a cell atlas using only lncRNA species. Overall, the MATQ-Drop platform permits the efficient characterization of synaptic heterogeneity and large-scale cell atlas construction. In the future, MATQ-Drop can be readily applied to other neurological and neurodegenerative diseases and shed new insights into understanding synaptic biology. It could be also used as a new tool to construct the brain connectome.

### Example 2

### Examples of Materials and Methods

### Microfluidic Device Design and Fabrication

The design and fabrication of the hydrogel bead generation device and the cell encapsulation device are previously described⁴³.

### Barcoded Bead Synthesis

The hydrogel bead production and barcode synthesis procedures were based on the work by Zilionis et al.⁴³. Two modifications were introduced in hydrogel bead production. First, the acrydite-modified oligonucleotide was designed to contain a deoxyUridine base, instead of a photocleavable moiety. Therefore, the primers can be released by the USER enzyme (NEB) instead of UV exposure. The step dim illumination is eliminated. Second, the concentration of the acrydite-modified DNA primer was reduced to 40 μM in the acrylamide-primer mix.

After hydrogel bead production, two rounds of split-and-pool were performed for barcode synthesis. In each round, the hydrogel beads were split into 144 wells; each well contained primers with a unique barcode as the template. Bst 2.0 warm-start DNA polymerase was used for barcode extension. The reaction was set at 55° C. for 3 h for the first round of split-and-pool, and 52° C. for 3 h for the second round. After each extension step, the reaction was quenched with a 1.5 volume of 25 mM EDTA, and leftover template oligonucleotides were denatured by alkaline and washed away following the protocol. Exonuclease I digestion was performed to remove primers with failed barcode extension.

### Cell Culture

HEK293T and NIH/3T3 cells were grown in DMEM/High Glucose medium (Gibco) with 10% fetal bovine serum (FBS, Gibco). Cell culture was passaged every 2-3 days.

### Mice

The C57BL/6 WT and 5×FAD mice were obtained from the Jackson Laboratory (Bar Harbor, ME). Mice were housed four per cage in a pathogen-free mouse facility with ad libitum access to food and water on a 12-hour light/dark cycle. Female mice were used for all experiments. All procedures were performed following National Institutes of Health (NIH) guidelines and approval of the Baylor College of Medicine Institutional Animal Care and Use Committee.

### Mouse Hippocampus Dissection

Around 9-month-old mice were deeply anesthetized with Ketamine (300 mg/kg)-Xylazine (30 mg/kg) solution, intraperitoneally (i.p), and perfused with saline. The brains were removed from the skull, and adult mice brain hemispheres were separated in half; the hippocampus was isolated from each hemisphere and immediately frozen in liquid nitrogen.

### Cell Line Nucleus Preparation

Cells were trypsinized and washed twice with phosphate-buffered saline (PBS). An equal number of HEK293T cells and NIH/3T3 cells were mixed, and then lysed into nuclei by incubating with the ice-cold Lysis Buffer (10 mM Tris-HCl, pH7.5, 10 mM NaCl, 3 mM MgCl₂, 0.1% NP-40, 0.1% Tween-20) on ice for 5 min. Before fixation, the nuclei were washed three times with Wash Buffer (10 mM Tris-HCl, pH7.5, 10 mM NaCl, 3 mM MgCl₂, 0.1% Tween-20). For each wash, the nuclei were first centrifuged at 500 g, 4° C. for 3 min, the supernatant was aspirated, and the nuclei pellet was resuspended in the Wash Buffer. After the third wash, we resuspended the nuclei in the Fixation Buffer (10 mM Tris-HCl, pH7.5, 10 mM NaCl, 3 mM MgCl₂, 0.2% Tween-20, 3% PFA) and incubated at room temperature for 10 min on an end-over-end rotator to fix the nuclei. Fixation was quenched by mixing with 3/20 volume of 2.5 M glycine. The fixed nuclei were washed twice with the Wash Buffer, and then passed through a 40 μm cell strainer.

Human Brain Nucleus Preparation from Frozen Samples

A protocol was followed that was developed by Krishnaswami et al.⁴⁴ to isolate the nuclei from the frozen brain samples. Briefly, the tissues were homogenized with Dounce homogenizer with 0.1% Triton X-100, followed by 3% PFA fixation at room temperature for 10 min. After quenching and washing away residual PFA, the homogenate was stained with Hoechst. Fluorescence-activated nucleus sorting (FANS) was performed to unbiasedly collect the Hoechst-positive single nuclei.

Human Brain Synaptosome Preparation from Frozen Samples

The method for synaptosome preparation is similar to nucleus preparation, but with two major differences: 1) Triton X-100 was omitted in the homogenization buffer; 2) Hoechst-negative population with a diameter smaller than 5 μm was sorted out by FACS. The detailed procedure is described as follows. First, ˜2 mm³ section of frozen brain tissues was chopped and rinsed in the homogenization buffer (250 mM sucrose, 25 mM KCl, 5 mM MgCl₂, 10 mM Tris-HCl pH 8.0, 1 μM DTT, 1× Halt protease inhibitor cocktail (ThermoFisher), 0.2 U/μl RNasein ribonuclease inhibitor (Promega)). The tissue was then transferred to the Dounce homogenizer (Wheaton), and homogenized by five strokes with the loose pestle, and ten strokes with the tight pestle. The homogenate was passed through a 40-μm cell strainer, and centrifuged at 1,500 g for 10 min at 4° C. The pellet was immediately resuspended in 25 mL of Fixation Buffer (10 mM Tris-HCl. pH7.5, 10 mM NaCl, 3 mM MgCl₂, 3% PFA), and incubated at room temperature for 10 min. Fixation was quenched by mixing with 3/20 volume of 2.5 M glycine. The fixed subneuronal structures were washed with Wash Buffer (10 mM Tris-HCl, pH7.5, 10 mM NaCl, 3 mM MgCl₂, 0.1% Tween-20) once, passed through another 40-μm cell strainer, and stained with Hoechst. FACS was then performed to enrich the Hoechst-negative synaptosome population smaller than 5 μm in diameter, calibrated using standard beads.

### Immunostaining of the Brain Synaptosomes

The fixed subneuronal structures were permeabilized with 0.2% Triton X-100 in PBS for 10 min on ice, and then pelleted by 3,000 g centrifugation at 4° C. for 5 min. Blocking of nonspecific was performed by incubating the samples with 5% BSA in PBS at room temperature for 30 min with rotation. The following primary antibodies were used for immunostaining: rabbit-anti-Synaptophysin (Invitrogen, MA5-14532, 1:60) and mouse-anti-PSD95 (Invitrogen, MA1-045, 1:400). Primary antibody binding was performed by 80-min-incubation with 0.5% BSA in PBS on an end-over-end rotor at room temperature. The samples were washed with 1 mL PBS with 0.5% BSA for 3 times. Secondary antibody binding was performed by 40-min-incubation with 0.5% BSA in PBS on an end-over-end rotor at room temperature, with the following secondary antibodies: goat-anti-rabbit-Alexa Fluor 647 (Invitrogen, A21244, 1:1667) and goat-anti-mouse-Cy3 (Invitrogen, A10521, 1:1667). The subneuronal structures were washed 3 times, stained with Hoechst 33342, and then proceeded with flow cytometry.

### Western Blot

To recover protein from fixed samples, we resuspended the samples in the Fixation Lysis Buffer (500 mM Tris-HCl. pH7.4, 2% SDS, 25 mM EDTA, 100 mM NaCl, 1% Triton X-100, 1% NP-40 and 1× Halt protease inhibitor cocktail (ThermoFisher)) and heated at 90° C. for 2 h. Protein concentration was quantified by Biorad DC Protein Assay, and 0.5 μg total protein was loaded for each Western blot using the standard protocol. The following primary antibodies were used in this study: synaptophysin (Invitrogen, MA5-14532, 1:200), synapsin-I (Cell Signaling Technology, 5297, 1:1000), CNPase (Millipore, MAB326R, 1:500), GFAP (Millipore, MAB360, 1:1000), and β-actin (Sigma-Aldrich, A1978, 1:2000).

### Permeabilization

Permeabilization of the PFA-fixed subcellular structures is required for efficient primer hybridization. To permeabilize the subcellular structures, we resuspended them with ice-cold PBS with 1% Triton X-100 and incubated them on ice for 5 min. The permeabilized subcellular structures were washed twice with ice-cold PBS containing 0.2% Triton X-100, and then adjusted to the concentration of ˜2,300 subcellular structures/μl before proceeding with reverse transcription.

### MATQ-Drop Procedure

### In Situ Reverse Transcription

For ˜25,000 subcellular structures, we prepared the following in situ reverse transcription mix: 4 μl 5× first strand buffer (Invitrogen), 1 μl 0.1 M DTT, 1 μl 1.8% Triton X-100, 0.5 μl 10 mM dNTP, 0.5 μl RNaseOUT (Invitrogen), 2 μl 11.5 μM MALBAC primer mix, 1 μl Superscript III reverse transcriptase (Invitrogen), and 11 μl fixed subcellular structures resuspended in PBS. Ten cycles of multiple annealing ramping from 8° C. to 50° C. were performed for efficient primer hybridization and reverse transcription.

### In Situ Poly a Tailing

The residual primers and any primer dimers were first washed away, and the subcellular structures were resuspended in 14.5 μl PBS with 0.2% Triton X-100. Next, 1 μl 1 mM dATP (mixed with 3 μM ddATP), 2 μl 10× terminal transferase buffer (NEB), 2 μl 2.5 mM CoCl₂, and 0.5 μl terminal transferase (NEB) were subsequently added to the subcellular structure suspension. The in situ polyA tailing reaction was incubated at 37° C. for 4 h, and quenched with 1.6 μl 0.5 M EDTA. In the reaction, we spiked in 1/333 of ddATP to prevent the polyA tail from growing too long, at the cost of losing 1−(332/333)²⁰=6% of the amplicons whose polyA tail is too short (<20) for efficient second strand synthesis.

### Barcoded Second Strand Synthesis

The fixed subcellular structures carrying polyA-tailed cDNA were washed. And individual subcellular structures were encapsulated with barcoded dT20 hydrogel beads and the 2× reaction mix using the microfluidic platform as previously described⁴³. After droplet encapsulation, the reaction was first incubated at 37° C. for 45 min to release the primers from the beads by USER enzyme (NEB); meanwhile, cDNA was released from RNA templates due to RNA digestion by RNase H (NEB) and RNase I_{f} (NEB) digestion. Next, 3 h incubation at 72° C. is performed to allow cDNA to diffuse out of the nucleus. Ten cycles were performed of [48° C. 2 min, 72° C. 1 min] to allow the barcoded dT20 primers to hybridize to the polyA tail of the released cDNA and Deep Vent (exo-) DNA polymerase (NEB) will initiate extension from the barcoded dT20 primers and accomplish second-strand synthesis. It is worth noting that this procedure does not involve a melting step, so each amplicon can only be converted to one double-strand DNA fragment.

### Post-Barcoding Cleanup

After the barcoded second strand synthesis was completed, the droplets emulsion was broken by mixing the emulsion with 1H,1H,2H,2H-Perfluoro-1-octanol (PFO, Sigma-Aldrich) in the presence of EDTA, which immediately quenches polymerase activity upon droplet breakage and therefore prevents barcode crosstalk. The remaining hydrogel beads in the aqueous phase were removed by centrifugation, and the supernatant was purified with 1× AMPure XP beads (Beckman) and eluted in 37.5 μl nucleus-free water.

ddTTP Sealing of Unused Bead Primers

To minimize barcode crosstalk in the amplification step, it is critical to quench the residual barcoded bead primers by ddTTP. The following ddTTP sealing mix was utilized: 37.5 μl purified product, 0.5 μl 10 mM ddTTP, 5 μl 10× terminal transferase buffer (NEB), 5 μl 2.5 mM CoCl₂, and 1 μl terminal transferase (NEB), and incubated at 37° C. for 3 h. The product was purified with 1× AMPure XP beads (Beckman) and eluted in 41 μl nucleus-free water.

### Library Amplification

PCR was performed to amplify 41 μl of the purified product by adding 5 μl 10× ThermoPol Buffer (NEB), 2.5 μl 10 μl M GAT27 primer (GTG AGT GAT GGT TGA GGA TGT GTG GAG), 1 μl 10 mM dNTP, and 0.5 μl Deep Vent (exo-) DNA polymerase (NEB). The following PCR program was used: 95° C. 2 min, 16-18 cycles of [95° C. 20 s, 63° C. 20s, 72° C. 2 min], and 72° C. 3 min. The amplified product was purified with 0.9× AMPure XP beads (Beckman), and the yield was quantified by Qubit (Invitrogen).

### Sequencing of MATQ-Drop Library

### Sequencing Library Preparation

For each MATQ-Drop library, 10 ng amplified product was mixed with 5 μl tagmentation DNA buffer (Illumina), 0.6 μl tagmentation DNA enzyme 2 (TDE2, Illumina), and the volume was brought up to 10 μl by adding nuclease-free water. The transposition mix was incubated at 55° C. for 15 min. Next, the reaction was quenched by adding 0.4 μl 0.5 M EDTA, and the transposase was released by 50° C. heating for 30 min.

To introduce the i5 index, the following 38.25 μl of reaction mix was prepared and added to each tube: 4 μl 10× ThermoPol Buffer (NEB), 2 μl 0.1 M MgSO₄, 1 μl 10 mM dNTP. 1.75 μl 10 μM Illumina Nextera N5XX indexed primer (AAT GAT ACG GCG ACC ACC GAG ATC TAC AC [i5 index] TCG TCG GCA GCG TC) (SEQ ID NO:1), 1.75 μl 10 μM MATQ-P700 primer (ACG TGT GCT CTT CCG ATC TCG CCG AAG ATG GTT GAG GAT GTG TGG AGA TA) (SEQ ID NO:2), 0.7 μl Deep Vent (exo-) DNA polymerase (NEB), and 28.8 μl nuclease-free water. The reaction was set on a thermal cycler with the following program: 65° C. 1 min, 72° C. 4 min, 95° C. 2 min, 7 cycles of [95° C. 20 s, 57° C. 30s, 72° C. 1 min], and 72° C. 2 min. The product was purified with 0.9× AMPure XP beads (Beckman), and eluted in 16 μl nuclease-free water.

To introduce the i7 index, we prepared the following PCR reaction: 16 μl pre-amplified product, 2 μl 10× ThermoPol Buffer (NEB), 0.5 μl 10 μM P5-22b primer (AAT GAT ACG GCG ACC ACC GAG A) (SEQ ID NO:3), 0.5 μl 10 μM P7-i7-MATQ indexed primer (CAA GCA GAA GAC GGC ATA CGA GAT [i7 index] GTG ACT GGA GTT CAG ACG TGT GCT CTT CCG ATC T) (SEQ ID NO:4), 0.4 μl 10 mM dNTP, and 0.3 μl Deep Vent (exo-) DNA polymerase (NEB). The reaction was set on a pre-heated thermal cycler with the following program: 95° C. 2 min, 5 cycles of [95° C. 20 s, 61° C. 20s, 72° C. 1 min], and 72° C. 2 min. The product was purified with 0.85× AMPure XP beads (Beckman), and eluted in 20 μl nuclease-free water.

### Sequencing

Libraries were pooled and quantified following the Illumina manual, and the pooled libraries were sequenced on the Illumina Nextseq 500 platform with 150 cycle sequencing kit. Custom Read 2 primer (CGC CGA AGA TGG TTG AGG ATG TGT GGA GAT A) (SEQ ID NO:5) was used following the Illumina manual. The sequencing cycles were either: Read 1:110 cycles; Index 1:6 cycles; Index 2:6 cycles; Read 2:45 cycles, or Read 1:76 cycles; Index 1:8 cycles; Index 2:8 cycles; Read 2:45 cycles.

### MATQ-Drop Raw Data Processing

The 3′ polyA tail of cDNA on Read 1 was trimmed with cutadapt⁴⁵ v3.1 paired read mode, with the read length filtering criteria: —minimum-length=30—pair-filter=any. Next, a custom Python script was used to assign the Read 2 cell barcode sequences to the pre-defined combination of Barcode1 and Barcode2 sequences with maximal two mismatches allowed for each segment of the barcode. Umi_tools⁴⁶ (v1.0.1) “extract” command was used to extract the reads with successfully assigned cell barcodes. Extracted Read 1 was mapped to the hg19 genome (or a combined genome of hg19 and mm10) with STAR⁴⁷ v2.5.3a, and the uniquely mapped reads with mapping scores no smaller than 250 were used for downstream analysis. The filtered reads were assigned to genes by featureCounts⁴⁸ v2.0.1 with appropriate Gencode annotation gtf files, and the assignment was based on transcript feature (-t transcript) with strandness (-s 2). For the reads with unambiguously assigned gene features, the umi_tools “count” command was used to generate the transcript-based digital gene expression matrix (parameter:—per-gene—gene-tag=XT—per-cell-method=directional).

To determine the cell barcodes that represent true nuclei instead of background crosstalk, we plotted out the (UMI counts) versus (barcode rank by UMI) plot, and the knee point was determined as the threshold for true nuclei (exemplified in FIG. 1b). Next, the cell barcodes representing true cells were used to generate the transcript-based gene expression matrix for true nuclei.

To generate the exon-based gene expression matrix, the inventors first filtered out the reads with unambiguously assigned transcript-based gene features. The inventors then reran featureCounts assignment with exon feature only (-t exon) and strandness (-s 2), followed by umi_tools count. The intron-based gene expression matrix was derived by subtracting the exon-based gene expression matrix from the transcript-based gene expression matrix.

### Clustering Analysis

### Data Filtering

Nuclei with mitochondrial UMI percentages higher than 5% were excluded for downstream analysis. In synaptome data, synapses with mitochondrial UMI percentages lower than 5% were excluded for downstream analysis. Then, mitochondrial and ribosomal genes were removed from the gene expression matrix. Low-quality nuclei with fewer than 200 intronic genes were excluded, and the nuclei with UMIs in the top 0.5% quantile were also removed. Low-quality Hoechst-negative subneuronal structures with fewer than 100 intronic genes were excluded, and those with UMIs in the top 0.5% quantile were also removed.

### Unsupervised Clustering

Standard Seurat4 integration pipeline with SCTransform normalization was used for clustering analysis^{49, 50}. Briefly, the intron-based (for nuclei), or the transcript-based (for synapses) gene expression matrix was normalized based on regularized negative binomial regression. Doublets were identified by the R package DoubletFinder⁵¹ with a stringently estimated doublet rate (5%). Next, datasets of different biological samples were integrated following the Seurat scRNA-seq integration vignette. Principal component analysis (PCA) and graph-based clustering were performed with the integrated data slot. Visualization of the clustering was accomplished with UMAP. Markers for each cluster were identified by the MAST⁵² algorithm embedded in the Seurat package with the following parameters: only.pos=TRUE, min.pct=0.25, logfc.threshold=0.5 for nuclei, or logfc.threshold=0.25 for synapses. Cell types were empirically assigned based on the overlap between cluster markers and canonical cell-type-specific markers. The above pipeline also applies to subclustering and lncRNA-based clustering analyses, except that the doublet identification and removal step was skipped because we only used the nuclei passing the “singlet” filter described above.

### Doublet Removal

Doublets were identified and removed by the R package DoubletFinder⁵¹ with a stringently estimated doublet rate (5%).

Markers for each cluster were identified by the MAST⁵² algorithm embedded in the Seurat package with the following parameters: only.pos=TRUE, min.pct=0.25, logfc.threshold=0.5. Cell types were empirically assigned based on the cluster markers and the expression of canonical cell-type-specific markers.

The same pipeline applies to subclustering, and lncRNA-based clustering analyses, except that the doublet identification and removal step was skipped because we only used the nuclei passing the “singlet” filter described above. For lncRNA-based clustering, only the top 1,000 variable features were used for PCA.

### Differentially Expressed Gene Analysis

For the cluster populations of interest, a pseudobulk count matrix was assembled for each biological sample by summarizing the total UMI counts. Next, bulk DEGs were identified with edgeR⁵³. A gene is defined as “differentially expressed” if abs (log₂(Fold Change))>log₂(1.3) and Benjamini-Hochberg FDR<0.05. It is worth noting that compared to the single-cell approach, the pseudobulk approach yields robust fold-change calculation when the two datasets show large differences in UMI detection, for example, nuclei versus synapses. The transcript-based gene expression matrix was used for DEG analysis among different subneuronal structures, while the exon-based gene expression matrix was used for DEG analysis between synapses and nuclei. Gene ontology enrichment analysis of the DEGs was performed using the Database for Annotation, Visualization, and Integrated Discovery (DAVID), and the inventors used the shared expressed genes (CPM>2) as the background list. Gene set enrichment analysis (GSEA) was performed on the log₂(CPM+1) matrix with the pseudobulk.

### Identification of Unspliced Genes

For each type of subcellular structure, a gene is defined as “expressed” if detected in at least 5% of the subcellular structures. For each neuron type, only the expressed genes shared by pre-synapses and nuclei were kept for analysis. The average intron percentages of the transcripts in pre-synapses (pct_intronsyn) and nuclei (pct_intron_{nucleus}) were computed respectively, and the splicing score (SS) at the synapse is defined as:

$$SS = \{ \begin{matrix}min ( \frac{{pct\_intron}_{syn} - {pct\_intron}_{nucleus}}{{pct\_intron}_{nucleus}} , 0 ) , & if {pct\_intron}_{nucleus} \neq 0 \\ 1 , & if {pct\_intron}_{nucleus} = 0\end{matrix}$$

For a transcript that is fully unspliced at the synapse, SS=0, while for a transcript that is fully spliced at the synapse, SS=1. For each neuronal type, the distribution shows a peak at 1, with a long tail towards 0. Therefore, we transform the SS into Z scores, and a gene is considered unspliced if splicing z score <−2.58 (equivalent to p value <0.01), and pct_intron_{nucleus}>0.25. The splicing score metrics were used in preranked GSEA.

### Data Availability

The raw sequencing files are available in Gene Expression Omnibus (GEO) database with accession number GEO: GSE199346.

### Code Availability

The analysis code customized for MATQ_Drop sequencing data is available at the Github website using Zonglab/MATQ_Drop.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.
1. Gupta, A., Wang, Y. & Markram, H. Organizing principles for a diversity of GABAergic interneurons and synapses in the neocortex. Science 287, 273-278 (2000).
2. Husi, H., Ward, M. A., Choudhary, J. S., Blackstock, W. P. & Grant, S. G. Proteomic analysis of NMDA receptor-adhesion protein signaling complexes. Nat Neurosci 3, 661-669 (2000).
3. Ibanez-Sandoval, O. et al. Electrophysiological and morphological characteristics and synaptic connectivity of tyrosine hydroxylase-expressing neurons in adult mouse striatum. J Neurosci 30, 6999-7016 (2010).
4. Cizeron, M. et al. A brainwide atlas of synapses across the mouse life span. Science 369, 270-275 (2020).
5. Zhu, F. et al. Architecture of the Mouse Brain Synaptome. Neuron 99, 781-799 e710 (2018).
6. Sheng, K., Cao, W., Niu, Y., Deng, Q. & Zong, C. Effective detection of variation in single-cell transcriptomes using MATQ-seq. Nat Methods 14, 267-270 (2017).
7. Macosko, E. Z. et al. Highly Parallel Genome-wide Expression Profiling of Individual Cells Using Nanoliter Droplets. Cell 161, 1202-1214 (2015).
8. Habib, N. et al. Massively parallel single-nucleus RNA-seq with DroNc-seq. Nat Methods 14, 955-958 (2017).
9. Zheng, G. X. et al. Massively parallel digital transcriptional profiling of single cells. Nat Commun 8, 14049 (2017).
10. Klein, A. M. et al. Droplet barcoding for single-cell transcriptomics applied to embryonic stem cells. Cell 161, 1187-1201 (2015).
11. Ramskold, D. et al. Full-length mRNA-Seq from single-cell levels of RNA and individual circulating tumor cells. Nat Biotechnol 30, 777-782 (2012).
12. Zong, C., Lu, S., Chapman, A. R. & Xie, X. S. Genome-wide detection of single-nucleotide and copy-number variations of a single human cell. Science 338, 1622-1626 (2012).
13. Hu, P. et al. Dissecting Cell-Type Composition and Activity-Dependent Transcriptional State in Mammalian Brains by Massively Parallel Single-Nucleus RNA-Seq. Mol Cell 68, 1006-1015 e1007 (2017).
14. Hafner, A. S., Donlin-Asp, P. G., Leitch, B., Herzog, E. & Schuman, E. M.

Local protein synthesis is a ubiquitous feature of neuronal pre- and postsynaptic compartments. Science 364 (2019).
15. Cajigas, I. J. et al. The local transcriptome in the synaptic neuropil revealed by deep sequencing and high-resolution imaging. Neuron 74, 453-466 (2012).
16. Hughes, A. N. & Appel, B. Oligodendrocytes express synaptic proteins that modulate myelin sheath formation. Nat Commun 10, 4125 (2019).
17. Koopmans, F. et al. SynGO: An Evidence-Based, Expert-Curated Knowledge Base for the Synapse. Neuron 103, 217-234 e214 (2019).
18. Caceres, A., Banker, G., Steward, O., Binder, L. & Payne, M. MAP2 is localized to the dendrites of hippocampal neurons which develop in culture. Brain Res 315, 314-318 (1984).
19. Naisbitt, S. et al. Shank, a novel family of postsynaptic density proteins that binds to the NMDA receptor/PSD-95/GKAP complex and cortactin. Neuron 23, 569-582 (1999).
20. Awasthi, A. et al. Synaptotagmin-3 drives AMPA receptor endocytosis, depression of synapse strength, and forgetting. Science 363 (2019).
21. Hines, J. H., Ravanelli, A. M., Schwindt, R., Scott, E. K. & Appel, B. Neuronal activity biases axon selection for myelination in vivo. Nat Neurosci 18, 683-689 (2015).
22. Mensch, S. et al. Synaptic vesicle release regulates myelin sheath number of individual oligodendrocytes in vivo. Nat Neurosci 18, 628-630 (2015).
23. Wake, H. et al. Nonsynaptic junctions on myelinating glia promote preferential myelination of electrically active axons. Nat Commun 6, 7844 (2015).
24. Sakers, K. et al. Astrocytes locally translate transcripts in their peripheral processes. Proc Natl Acad Sci USA 114, E3830-E3838 (2017).
25. Stuart, T. et al. Comprehensive Integration of Single-Cell Data. Cell 177, 1888-1902 e1821 (2019).
26. Lake, B. B. et al. Neuronal subtypes and diversity revealed by single-nucleus RNA sequencing of the human brain. Science 352, 1586-1590 (2016).
27. Buckley, P. T. et al. Cytoplasmic intron sequence-retaining transcripts can be dendritically targeted via ID element retrotransposons. Neuron 69, 877-884 (2011).
28. Glanzer, J. et al. RNA splicing capability of live neuronal dendrites. Proc Natl Acad Sci USA 102, 16859-16864 (2005).
29. Bell, T. J. et al. Cytoplasmic BK (Ca) channel intron-containing mRNAs contribute to the intrinsic excitability of hippocampal neurons. Proc Natl Acad Sci USA 105, 1901-1906 (2008).
30. Bell, T. J. et al. Intron retention facilitates splice variant diversity in calcium-activated big potassium channel populations. Proc Natl Acad Sci USA 107, 21152-21157 (2010).
31. Aoto, J., Martinelli, D. C., Malenka, R. C., Tabuchi, K. & Sudhof, T. C. Presynaptic neurexin-3 alternative splicing trans-synaptically controls postsynaptic AMPA receptor trafficking. Cell 154, 75-88 (2013).
32. Jones, K. A. et al. Neurodevelopmental disorder-associated ZBTB20 gene variants affect dendritic and synaptic structure. PLOS One 13, e0203760 (2018).
33. Frese, C. K. et al. Quantitative Map of Proteome Dynamics during Neuronal Differentiation. Cell Rep 18, 1527-1542 (2017).
34. Dougherty, M. K. et al. KSR2 is a calcineurin substrate that promotes ERK cascade activation in response to calcium signals. Mol Cell 34, 652-662 (2009).
35. Hong, S. et al. Complement and microglia mediate early synapse loss in Alzheimer mouse models. Science 352, 712-716 (2016).
36. Roy, E. R. et al. Type I interferon response drives neuroinflammation and synapse loss in Alzheimer disease. J Clin Invest 130, 1912-1930 (2020).
37. Shankar, G. M. et al. Natural oligomers of the Alzheimer amyloid-beta protein induce reversible synapse loss by modulating an NMDA-type glutamate receptor-dependent signaling pathway. J Neurosci 27, 2866-2875 (2007).
38. Mathys, H. et al. Single-cell transcriptomic analysis of Alzheimer's disease. Nature 570, 332-337 (2019).
39. Habib, N. et al. Disease-associated astrocytes in Alzheimer's disease and aging. Nat Neurosci 23, 701-706 (2020).
40. Zhou, Y. et al. Human and mouse single-nucleus transcriptomics reveal TREM2-dependent and TREM2-independent cellular responses in Alzheimer's disease. Nat Med 26, 131-142 (2020).
41. Zalcman, G., Federman, N. & Romano, A. CaMKII Isoforms in Learning and Memory: Localization and Function. Front Mol Neurosci 11, 445 (2018).
42. Liu, S. J. et al. Single-cell analysis of long non-coding RNAs in the developing human neocortex. Genome Biol 17, 67 (2016).
43. Zilionis, R. et al. Single-cell barcoding and sequencing using droplet microfluidics. Nat Protoc 12, 44-73 (2017).
44. Krishnaswami, S. R. et al. Using single nuclei for RNA-seq to capture the transcriptome of postmortem neurons. Nat Protoc 11, 499-524 (2016).
45. Martin, M. Cutadapt removes adapter sequences from high-throughput sequencing reads. EMBnet J 17, 3 (2011).
46. Smith, T., Heger, A. & Sudbery, I. UMI-tools: modeling sequencing errors in Unique Molecular Identifiers to improve quantification accuracy. Genome Res 27, 491-499 (2017).
47. Dobin, A. et al. STAR: ultrafast universal RNA-seq aligner. Bioinformatics 29, 15-21 (2013).
48. Liao, Y., Smyth, G. K. & Shi, W. featureCounts: an efficient general purpose program for assigning sequence reads to genomic features. Bioinformatics 30, 923-930 (2014).
49. Hao, Y. et al. Integrated analysis of multimodal single-cell data. Cell 184, 3573-3587 e3529 (2021).
50. Hafemeister, C. & Satija, R. Normalization and variance stabilization of single-cell RNA-seq data using regularized negative binomial regression. Genome Biol 20, 296 (2019).
51. McGinnis, C. S., Murrow, L. M. & Gartner, Z. J. DoubletFinder: Doublet Detection in Single-Cell RNA Sequencing Data Using Artificial Nearest Neighbors. Cell Syst 8, 329-337 e324 (2019).
52. Finak, G. et al. MAST: a flexible statistical framework for assessing transcriptional changes and characterizing heterogeneity in single-cell RNA sequencing data. Genome Biol 16, 278 (2015).
53. Robinson, M. D., McCarthy, D. J. & Smyth, G. K. edgeR: a Bioconductor package for differential expression analysis of digital gene expression data. Bioinformatics 26, 139-140 (2010).

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method of producing a library representing RNA related to a subcellular compartment or structure, comprising the steps of:
(a) fixing cellular material that is or comprises one or more subcellular compartments or structures such that RNA associated with the structure is affixed to the structure;
(b) subjecting the subcellular compartments or structures and the RNA to first primers to generate a collection of first complementary polynucleotides that are complementary to one or more different regions in the RNA, thereby producing hybrid molecules between the RNA and first complementary polynucleotides, said hybrid molecules being associated with the subcellular compartments or structures, wherein the first primers comprise random sequence, or random sequence of only three types of nucleotides, or random sequence of only two types of nucleotides, and an adaptor;
(c) generating a common tail sequence on a 3′ end of the first complementary polynucleotides in the hybrid molecules, wherein said common tail sequence is complementary to a second primer;
(d1) encapsulating the subcellular compartments or structures and RNA in microscopic volume or microscope volume compartments with particles comprising associated therewith the second primers that also comprise one or more unique molecular identifier sequences (UMI) and one or more barcodes comprising known sequence that enables pooling of desired polynucleotides;
releasing the primers from the beads; or
(d2) exposing the hybrid molecules to a substrate comprising the second primers that also comprise one or more unique molecular identifier sequences (UMI) and one or more barcodes comprising known sequence that enables pooling of desired polynucleotides;
release cDNA from the structure;
(e) producing second strand synthesis upon hybridization of at least part of the second primer to the tail of the first complementary polynucleotides, thereby producing second complementary polynucleotides comprising at least part of the RNA sequence, the UMI, and the barcode; and
(f) optionally amplifying the second complementary polynucleotide.

2. The method of claim 1, wherein a plurality of second complementary polynucleotides are amplified and/or sequenced.

3. The method of claim 2, wherein the second complementary polynucleotides are amplified to produce amplified second complementary polynucleotides, followed by sequencing of one or more of the amplified second complementary polynucleotides.

4. The method of claim 2 or 3, wherein the amplifying is by polymerase chain reaction or one or more isothermal amplification methods.

5. The method of any one of claims 2-4, wherein the amplifying is by polymerase chain reaction, one or more isothermal amplification methods, or one or more linear amplification methods.

6. The method of claim 5, wherein the amplifying is by polymerase chain reaction followed by next-generation sequencing.

7. The method of any one of claims 1-6, wherein the cellular material for fixing is fresh, frozen, or was previously frozen.

8. The method of claim 7, wherein the fixing comprises subjecting the cellular material to about 0.1% to 100% paraformaldehyde.

9. The method of any one of claims 1-8, wherein following the fixing step, the subcellular compartments or structures are enriched.

10. The method of claim 9, wherein the subcellular compartments or structures are enriched by flow cytometry or density gradient centrifugation.

11. The method of any one of claims 1-10, wherein following the fixing step, the subcellular compartments or structures are permeabilized.

12. The method of claim 11, wherein the subcellular compartments or structures are permeabilized by one or more surfactants.

13. The method of any one of claims 1-12, wherein the subcellular compartment or structure is a synaptosome, nucleus, mitochondria, plastid, lysosome, ribosome, lysosome, endoplasmic reticulum, Golgi apparatus, dendrites, axons, synapses, node of Ranvier, dendritic spine, axon initial segment), synaptic terminal, dendritic spine, or extracellular vesicle.

14. The method of any one of claims 1-13, wherein the common tail sequence is a homopolymeric sequence.

15. The method of claim 14, wherein the homopolymeric sequence was added to the 3′ end of the first complementary polynucleotides by terminal transferase.

16. The method of claim 14 or 15, wherein the homopolymeric sequence comprises adenosines, and the second primers at least comprise thymosines.

17. The method of any one of claims 1-16, wherein the common tail sequence is added to the 3′ end of the first complementary polynucleotides by template switching activity of reverse transcriptase.

18. The method of any one of claims 1-17, wherein the microscopic volume is microliter, nanoliter, picoliter, or femtoliter volumes.

19. The method of any one of claims 1-18, wherein the microscopic volume or microscope volume compartments comprises droplets.

20. The method of claim 19, wherein the droplets are in microwells.

21. The method of any one of claims 1-20, wherein the cDNA is released from the subcellular structures by a stimulus.

22. The method of claim 21, wherein the stimulus comprises heating, pH changes, and/or enzymatic cleavage.

23. The method of claim 22, wherein the enzymatic cleavage is RNAse H, RNase I, or both.

24. The method of claim 1, wherein in (d2), the second primers are region-specific with respect to spatial resolution of the subcellular structure.

25. The method of any one of claims 1-24, wherein the primers are attached to the beads by a linker or by a covalent bond.

26. The method of any one of claims 1-24, wherein the primers are released from the particles enzymatically, chemically, and/or physically.

27. The method of claim 26, wherein the chemical release is by ultraviolet radiation and/or a reducing agent.

28. The method of claim 26 or 27, wherein the physical release is from heating.

29. The method of any one of claims 1-26, wherein one or more of the first primers bind to intronic sequences in nascent RNA.

30. The method of any one of claims 1-26, wherein one or more of the first primers bind to long non-coding RNA.

31. The method of claim 28, wherein the long non-coding RNA comprises a polyadenylated tail.

32. The method of claim 28, wherein the long non-coding RNA lacks a polyadenylated tail.

33. The method of any one of claims 1-32, wherein the RNA associated with the structure comprises nascent RNA, microRNA, long non-coding RNA, and/or mRNA.


SEQUENCE LISTING (10 sequences)

SEQ ID NO: 1 - DNA, 29 bp, synthetic construct
  source 1..29: mol_type = other DNA
aatgatacggcgaccaccgagatctacac

SEQ ID NO: 2 - DNA, 50 bp, synthetic construct
  source 1..50: mol_type = other DNA
acgtgtgctcttccgatctcgccgaagatggttgaggatgtgtggagata

SEQ ID NO: 3 - DNA, 22 bp, synthetic construct
  source 1..22: mol_type = other DNA
aatgatacggcgaccaccgaga

SEQ ID NO: 4 - DNA, 24 bp, synthetic construct
  source 1..24: mol_type = other DNA
caagcagaagacggcatacgagat

SEQ ID NO: 5 - DNA, 31 bp, synthetic construct
  source 1..31: mol_type = other DNA
cgccgaagatggttgaggatgtgtggagata

SEQ ID NO: 6 - DNA, 20 bp, synthetic construct
  source 1..20: mol_type = other DNA
tttttttttttttttttttt

SEQ ID NO: 7 - DNA, 27 bp, synthetic construct
  source 1..27: mol_type = other DNA
gtgagtgatggttgaggatgtgtggag

SEQ ID NO: 8 - DNA, 13 bp, synthetic construct
  source 1..13: mol_type = other DNA
aaaaaaaaaaaaa

SEQ ID NO: 9 - DNA, 14 bp, synthetic construct
  source 1..14: mol_type = other DNA
tcgtcggcagcgtc

SEQ ID NO: 10 - DNA, 34 bp, synthetic construct
  source 1..34: mol_type = other DNA
gtgactggagttcagacgtgtgctcttccgatct